(19) United States
(12) Patent Application Publication
(10) Pub. No.: US 2023/0382820 A1
(43) Pub. Date: Nov. 30, 2023

(54) PRODUCTION OF LIQUID HYDROCARBONS FROM CARBON DIOXIDE, IN COMBINATION WITH HYDROGEN OR A HYDROGEN SOURCE

(71) Applicant: GTI Energy, Des Plaines, IL (US)

(72) Inventors: Terry MARKER, Park Ridge, IL (US); Jim WANGEROW, Oak Park, IL (US); Martin LINCK, Wilmette, IL (US)

(21) Appl. No.: 18/200,026

(22) Filed: May 22, 2023

Related U.S. Application Data
(60) Provisional application No. 63/344,599, filed on May 22, 2022.

(51) Int. Cl. C07C 1/04; C10G 55/06; C01B 3/16; B01J 23/42; B01J 23/10; B01D 3/14

(52) U.S. Cl. CPC C07C 1/0435; C10G 55/06; C01B 3/16; B01J 23/42; B01J 23/10; B01D 3/14; C10G 2400/04; C10G 2400/08; C10G 2400/30; C01B 2203/0283; C01B 2203/062; C01B 2203/107; C01B 2203/1082

(57) ABSTRACT

Pathways are disclosed for the production of liquid hydrocarbon products comprising gasoline and/or diesel boiling-range hydrocarbons, and in certain cases renewable products having non-petroleum derived carbon. In representative processes, a gaseous feed mixture comprising CO₂ in combination H₂ and/or CH₄ (or other hydrocarbon source of H₂) is converted by reforming and/or reverse water-gas shift (RWGS) reactions, optionally further in combination with Fischer-Tropsch (FT) synthesis and/or cracking. A preferred gaseous feed mixture comprises biogas or otherwise a mixture of CO₂ and H₂ that is not readily upgraded using conventional processes. Catalysts described herein have a high activity for catalyzing the reforming (including dry reforming) of CH₄ and other light hydrocarbons (e.g., those having been produced via FT synthesis and recycled as light ends back to the process) as well as simultaneously catalyzing the RWGS reaction. These attributes allow for flexibility in terms of compositions that may be converted efficiently. Economics of small-scale operations may be improved, if necessary, using an electrically heated reforming reactor in the first or initial reforming stage or RWGS stage.

### CROSS-REFERENCE TO RELATED APPLICATIONS

This application claims priority to U.S. provisional application no. 63/344,599, filed May 22, 2022, which is incorporated by reference in its entirety.

### FIELD OF THE INVENTION

Aspects of the invention relate to processes and associated catalysts for producing, from gaseous feed mixtures comprising carbon dioxide (CO₂), liquid hydrocarbons including naphtha boiling-range hydrocarbons, jet fuel boiling-range hydrocarbons, and/or diesel boiling-range hydrocarbons. The processes utilize one or more reactions of reforming (including CO₂ and/or steam reforming), reverse water-gas shift (RWGS), and Fischer-Tropsch (FT) synthesis, optionally in combination with wax cracking and/or isomerization.

### DESCRIPTION OF RELATED ART

The ongoing search for alternatives to crude oil, as a conventional source of carbon for hydrocarbon products, is increasingly driven by a number of factors. These include diminishing petroleum reserves, higher anticipated energy demands, and heightened concerns over greenhouse gas (GHG) emissions from sources of non-renewable carbon, which here refers to fossil carbon. Hydrocarbon products of greatest industrial significance and interest, in terms of having their carbon content replaced with non-petroleum derived carbon, include transportation and heating fuels as well as precursors for specialty chemicals. Liquid hydrocarbons, i.e., hydrocarbons that are liquid at room temperature, are representative of these hydrocarbon products.

Carbon dioxide (CO₂) is a major contributor to GHG emissions and is found in gases generated from combustion as performed in engines, electricity production, and both commercial and residential heating. In general, a great number of small- and large-scale processes produce waste gases containing CO₂ that is derived from the crude oil-based hydrocarbon products described above. In some cases, CO₂ may be obtained as a component of a mixture of gases including hydrogen (H₂) and/or methane (CH₄), in which the CO₂ may or may not be a combustion product. Examples of such mixtures include industrial off gases obtained from the production of H₂ by the reforming of CH₄, in which the CO₂ is used as a reactant (in the case of dry reforming) and/or is generated by the water-gas shift reaction. In addition, sources of natural gas, while predominantly methane, may also include a significant content of CO₂ that is extracted in this resource. Other gaseous feed mixtures of CO₂ with CH₄ include those in which the latter component is a renewable resource, such as in the specific case of (i) biogas obtained from anaerobic bacterial digestion of biowastes or from wastewater treatment, (ii) gaseous products of biomass conversion (e.g., biomass gasification, pyrolysis, or hydropyrolysis, such as in the case of supercritical water gasification of biomass), (iii) landfill gases, or (iv) gaseous products of the electrochemical reduction of carbon dioxide.

In view of its abundance in natural gas reserves and oil-associated gases, methane has become the focus of a number of possible synthesis routes. Currently, natural gas is the most underutilized of fossil resources, and it is frequently flared (combusted) in large quantities, particularly in the case of “stranded” natural gas or other sources that are too isolated and/or lacking in quantity, rendering their transport to large-scale processing facilities an uneconomical proposition. In addition, fracking technology has resulted in decreasing prices of natural gas in the U.S., with an increasing supply of this resource globally. Moreover, methane is one of the most common products that can be produced from renewable resources, and particularly those obtained from the processing of biowastes and biomass, as well as other resources as noted above. Therefore, the conversion of methane, and especially methane that is obtained from “renewable carbon,” which here refers to non-fossil carbon (including, for example, atmospheric CO₂ and carbon derived from biomass), including sources such as biogas, represents an area of considerable interest for development on the industrial scale with favorable economics.

A key commercial process for converting methane into fuels involves a first conversion step to produce synthesis gas (syngas), followed by a second, downstream Fischer-Tropsch (FT) conversion step. With respect to the first conversion step, upstream of FT, known processes for the production of syngas from methane include partial oxidation reforming, based on the highly exothermic oxidation of methane with oxygen, and autothermal reforming (ATR), based on a combination of the highly exothermic oxidation of methane with oxygen and the highly endothermic reforming of steam and methane and the mildly endothermic water gas shift, to yield a process that has net enthalpy near zero. Steam methane reforming (SMR), in contrast, uses steam as the oxidizing agent, such that the thermodynamics are significantly different, not only because the production of steam itself can require an energy investment, but also because reactions involving methane and water are highly endothermic. The SMR reaction proceeds according to:

CH₄+H₂O→CO+3H₂.

More recently, it has also been proposed to use carbon dioxide as the oxidizing agent for methane, such that the desired syngas is formed by the reaction of carbon in its most oxidized form with carbon in its most reduced form, according to:

CH₄+CP₂→2CO+2H₂.

This reaction has been termed the “dry reforming” of methane, and because it is highly endothermic, thermodynamics for the dry reforming of methane are less favorable compared to ATR or even SMR. However, the stoichiometric consumption of one mole of carbon dioxide per mole of methane has the potential to reduce the overall carbon footprint of liquid fuel production, providing a “greener” consumption of methane. This CO₂ consumption rate per mole of feed increases in the case of reforming higher hydrocarbons (e.g., C₂-C₆ paraffins), which may be desired, for example, if hydrogen production (e.g., for refinery processes) is the objective. In any event, the thermodynamic barrier remains a major challenge and relates to the fact that CO₂ is completely oxidized and very stable, such that significant energy is needed for its activation as an oxidant. In view of this, a number of catalyst systems have been investigated for overcoming the activation energy barrier for the dry reforming of methane, and these are summarized, for example, in a review by Lavoie (FRONTIERS IN CHEMISTRY (Nov. 2014), Vol. 2 (81): 1-17), identifying heterogeneous catalyst systems as being the most popular in terms of catalytic approaches for carrying out this reaction.

Whereas nickel-based catalysts have shown effectiveness in terms of lowering the activation energy for the above dry reforming reaction, a high rate of carbon deposition (coking) of these catalysts has also been reported in Lavoie. The undesired conversion of methane to elemental carbon can proceed through methane cracking (CH₄→C+2H₂) or the Boudouard reaction (2CO→C+CO₂) at the reaction temperatures typically required for the dry reforming of methane. More recently, other types of catalysts, including those comprising noble metals on a ceria-containing support, have been described in U.S. Pat. No. 10,738,247; U.S. Pat. No. 10,906,808; US 2020/0087144; and US 2020/0087576, assigned to Gas Technology Institute (Des Plaines, IL). Such catalysts have been demonstrated to exhibit high activity and stability (low coking rate) in reforming based on CO₂ alone or a combination of CO₂ and steam. In addition, the high tolerance to sulfur-bearing contaminants (e.g., H₂S), exhibited by these catalysts, can further improve process economics in terms of lowering costs normally associated with feed pretreatment.

With respect to the second step involving FT conversion, synthesis gas containing a mixture of hydrogen and carbon monoxide (CO) is subjected to successive cleavage of C—O bonds and formation of C—C bonds with the incorporation of hydrogen. This mechanism provides for the formation of hydrocarbons, and particularly straight-chain alkanes with a distribution of molecular weights that can be controlled to some extent by varying the FT reaction conditions (temperature and feed CO:H₂ ratio) and catalyst properties. Such properties include pore size and other characteristics of the support material. The choice of catalyst can impact FT product yields in other respects. For example, iron-based FT catalysts tend to produce more oxygenates, whereas ruthenium as the active metal tends to produce exclusively paraffins. The reaction pathways of FT synthesis follow a statistical kinetic model, which leads to hydrocarbons having an Anderson-Schultz-Flory distribution of their carbon numbers. The conversion level can be appropriately tuned to favor production of hydrocarbons having desired molecular weights, although lower- and higher-carbon number hydrocarbons invariably constitute part of the FT product slate. Overall, the state of the art would benefit from technologies for the efficient conversion of industrially available gaseous mixtures containing CO₂ with other beneficial reactants such as H₂ and/or CH₄, to products comprising liquid hydrocarbons, for example those that may be characterized as naphtha boiling-range, jet fuel boiling-range, or diesel boiling-range hydrocarbons.

### SUMMARY OF THE INVENTION

Aspects of the invention are associated with the discovery of processes in which CO₂, which is recognized as an undesirable atmospheric pollutant contributing to climate change, can be effectively utilized for its carbon content in the production of valuable hydrocarbons, including liquid hydrocarbons useful as transportation fuels. In this manner, CO₂ can advantageously serve to displace hydrocarbon fuels refined from petroleum and other fossil-derived sources. Whereas technologies are available for concentrating CO₂ from air, such as via direct air capture routes, the present invention provides a viable option for utilizing air-extracted CO₂ to produce hydrocarbons, which can be implemented on an industrial scale. This contrasts, for example, with CO₂ sequestration, which is relatively expensive and limited in terms of its capacity.

In this regard, the present invention relates to novel pathways for the production of C₄⁺ hydrocarbons (e.g., separated and optionally recovered fractions comprising or consisting of naphtha boiling-range hydrocarbons, jet fuel boiling-range hydrocarbons, and/or diesel boiling-range hydrocarbons), i.e., in which some or all (e.g., at least about 70%) of their carbon content (whether expressed on a wt-% or mole-% basis) is not derived from petroleum, such as in the case of this carbon content being renewable. Advantageously, whether or not the carbon content is renewable carbon, at least a portion (e.g., at least about 20%, at least about 30%, or at least about 40%), of the total carbon content of representative liquid hydrocarbon products (or of C₄⁺ hydrocarbons contained in these products, or of specific boiling-range fractions separated and optionally recovered from these products) described herein may be derived from CO₂, for example being present initially in a gaseous feed mixture, and being optionally extracted from air (e.g., via direct air capture). In the case of a renewable carbon content that is also derived from CO₂, such CO₂ may be obtained, for example, from biogas (i.e., such CO₂ is originally contained in biogas) or from a gas resulting from the decomposition, combustion, or gasification of biomass. In the case of a non-renewable carbon content that is derived from CO₂, such CO₂ may be obtained, for example, as a fossil fuel combustion product. In any of these cases, and in general, whether CO₂ is obtained from (i) air, (ii) a renewable carbon source such as biomass (e.g., which produces biogas), and/or (iii) an industrial waste gas such as a combustion product, it can be appreciated that CO₂ used to provide at least a portion of the carbon content is beneficially utilized in the production of C₄⁺ hydrocarbons, rather than remaining in, or being released into, the atmosphere.

Optionally in combination with having a carbon content derived from CO₂, including renewable CO₂, to an extent as described above, representative liquid hydrocarbon products described herein, including C₄⁺ hydrocarbons contained in these products and specific boiling-range fractions separated and optionally recovered from these products, may have a hydrogen content that is derived at least in part from: (a) “electrolysis hydrogen” which here refers to hydrogen produced via electrolysis, optionally utilizing renewable electricity such as derived from solar, wind, nuclear, or hydro energy, (b) “fossil hydrogen with carbon capture and sequestration (CCS)” which here refers to hydrogen produced via coal gasification or natural gas reforming in combination with carbon capture and sequestration, (c) “bio-gasification hydrogen” which here refers to hydrogen produced via biomass gasification; and (d) “methane pyrolysis hydrogen” which here refers to hydrogen produced via methane pyrolysis. For example, processes described herein and utilizing electrolysis hydrogen, fossil hydrogen with CCS, bio-gasification hydrogen, or methane pyrolysis hydrogen in a fresh makeup Hz-containing feed, may be used to produce these products described herein, including such C₄⁺ hydrocarbons and such specific boiling-range fractions. At least a portion of the total hydrogen content of these products may therefore be derived from electrolysis hydrogen, fossil hydrogen with CCS, bio-gasification hydrogen, or methane pyrolysis hydrogen.

Accordingly, representative embodiments of the invention are directed to C₄⁺ hydrocarbon fractions, including those separated and optionally recovered C₄⁺ hydrocarbon fractions as described herein, for example liquid fractions separated in, and optionally recovered from, processes as described herein. Such C₄⁺ hydrocarbon fractions may comprise naphtha boiling-range hydrocarbons, jet fuel boiling-range hydrocarbons and/or diesel boiling-range hydrocarbons, for example a given C₄⁺ hydrocarbon fraction may comprise substantially all (e.g., greater than about 95 wt-%) of any one of naphtha boiling-range hydrocarbons, jet fuel boiling-range hydrocarbons, or diesel boiling-range hydrocarbons, or otherwise may consist of, or consist essentially of, hydrocarbons within such boiling ranges. According to particular embodiments, (i) at least about 20%, at least about 50%, at least about 80%, or at least about 95%, of a total carbon content of any of such liquid hydrocarbon product or C₄⁺ hydrocarbon fraction described herein may be derived from atmospheric CO₂ and/or biogas CO₂ and/or CO₂ in a gas resulting from the decomposition, combustion, or gasification of biomass and/or (ii) at least about 20%, at least about 50%, at least about 80%, or at least about 95%, of a total hydrogen content of any such liquid hydrocarbon product or C₄⁺ hydrocarbon product described herein may be derived from electrolysis hydrogen, fossil hydrogen with CCS, bio-gasification hydrogen, or methane pyrolysis hydrogen. Importantly, liquid hydrocarbon products produced by processes described herein, as well as C₄⁺ hydrocarbon fractions separated in, and optionally recovered from, processes described herein may have (i) a carbon content at least partially derived, and possibly substantially completely derived, from atmospheric CO₂ (e.g., obtained from direct air capture) and/or biogas CO₂ and/or CO₂ in a gas resulting from the decomposition, combustion, or gasification of biomass and/or (ii) a hydrogen content at least partially derived, and possibly substantially completely derived, from electrolysis hydrogen (e.g., obtained from solar- and/or wind-generated electricity), fossil hydrogen with CCS, bio-gasification hydrogen, or methane pyrolysis hydrogen. These products and fractions may therefore be associated with processes as described herein, in which atmospheric CO₂ and/or biogas CO₂ and/or CO₂ in a gas resulting from the decomposition, combustion, or gasification of biomass may be present in a fresh makeup CO₂ and/or CH₄-containing feed (e.g., such feed may comprise, substantially comprise, consist of, or consist essentially of, atmospheric CO₂ and/or biogas CO₂ and/or CO₂ in a gas resulting from the decomposition, combustion, or gasification of biomass), as an input to such processes, and/or electrolysis hydrogen, fossil hydrogen with CCS, bio-gasification hydrogen, or methane pyrolysis hydrogen may be present in a fresh makeup H₂-containing feed (e.g., such feed may comprise, substantially comprise, consist of, or consist essentially of, electrolysis hydrogen, fossil hydrogen with CCS, bio-gasification hydrogen, or methane pyrolysis hydrogen), as an input to such processes. Any C₄⁺ hydrocarbon fraction, and particularly any recovered C₄⁺ hydrocarbon fraction, may be an output of such processes.

Further aspects of the invention are associated with the discovery that common sources of CO₂, and especially gaseous mixtures of CO₂, in combination with H₂ and/or a source of H₂ (i.e., a hydrogen source such as CH₄, C₂H₆, C₃H₈ and/or H₂O), can be used efficiently as feeds in producing liquid hydrocarbon products. Importantly, the whole feed and therefore all of these components may be reactants in one or more reactions of reforming (including CO₂ and/or steam reforming), reverse water-gas shift (RWGS), and Fischer-Tropsch (FT) synthesis, optionally in combination with wax cracking and/or isomerization, which are used to obtain C₄⁺ hydrocarbons. In the case of a gaseous feed mixture comprising both CH₄ (as a hydrogen source) and CO₂, e.g., a gaseous feed mixture that is biogas or that comprises biogas, these components may be reacted in a reforming stage, according to the dry reforming reaction above, to produce a synthesis gas intermediate comprising H₂ and CO, i.e., an H₂/CO mixture). This intermediate may, in turn, be converted to the liquid hydrocarbon product, at least partially via FT synthesis, and optionally via a combination of FT synthesis and wax cracking. The latter reaction may be used to adjust the carbon number distribution of hydrocarbons otherwise obtained from FT synthesis alone, and especially to convert a wax fraction (e.g., comprising normal C₂₀⁺ hydrocarbons) otherwise contained in an FT synthesis effluent (in the absence of cracking), to normal or branched C₄-C₁₉ hydrocarbons that contribute to the yield of liquid hydrocarbons from the process (e.g., liquid hydrocarbons present in recovered C₄⁺ hydrocarbon fractions).

In the case of a gaseous feed mixture comprising both H₂ and CO₂, e.g., a gaseous feed mixture that is or comprises an industrial off gas, such as a tail gas (or equivalently off gas) of a pressure swing absorber (PSA) used to purify H₂ produced by a steam methane reforming, or that is or comprises a mixture of atmospheric CO₂ and/or biogas CO₂ and/or CO₂ in a gas resulting from the decomposition, combustion, or gasification of biomass, in combination with a mixture of electrolysis hydrogen, fossil hydrogen with CCS, bio-gasification hydrogen, or methane pyrolysis hydrogen, the H₂ and CO₂ may be reacted according to the RWGS reaction to produce a synthesis gas intermediate for conversion to a liquid hydrocarbon product as described above, at least partially via FT synthesis, and optionally via a combination of FT synthesis and wax cracking. As is known in the art, a PSA tail gas used to purify H₂ produced by stream methane reforming is a byproduct obtained from the production of H₂ by the reforming of CH₄ and steam Simultaneously with the RWGS reaction, in the event that CH₄ is present in the H₂ and CO₂ gas mixture, CH₄ and CO₂ may be reacted according to the dry reforming reaction above, thereby adding to the yield of H₂ and CO in the synthesis gas intermediate, with CO having been obtained from a combination of both RWGS and dry reforming.

Accordingly, other aspects of the invention are associated with the discovery that catalysts described herein, having a high activity for catalyzing the reforming (including dry reforming) of CH₄ and/or other hydrogen sources, such as C₂H₆, and/or C₃H₈, are likewise effective, under the same conditions, for catalyzing the RWGS reaction. These attributes of such catalysts are therefore advantageous in producing liquid hydrocarbon products, particularly from gaseous feed mixtures, as described herein, comprising CO₂ together with H₂ and/or a source of H₂ (i.e., a hydrogen source such as CH₄, C₂H₆, C₃H₈ and/or H₂O), all of which components may be beneficially utilized as reactants in these reactions. Such gaseous mixtures may otherwise be difficult to monetize and/or may conventionally be combusted for their heating value. According to some embodiments, for example those involving the processing of gaseous mixtures on a relatively small scale, the use of an electrically heated reforming reactor in the first or initial stage (e.g., a reforming stage or an RWGS stage) to perform one or both of these reactions may further improve processing efficiency and equipment compactness, leading to reduced costs. Small scale operations may involve, for example, the processing of gaseous feed mixtures obtained from lower capacity biogas production facilities or stranded gas reserves. An electrically heated reforming reactor may include one or more resistive or inductive heating elements for internally and/or externally heating the reforming reactor and thereby effectively controlling localized and overall heat input into a bed of reforming/RWGS catalyst as described herein. Representative electrically heated reforming reactors thereby provide precise (e.g., axial and/or radial) and responsive bed temperature control, and examples of these are described in co-pending U.S. application Ser. No. 17/402,865, published as US 2022/0134298 and hereby incorporated by reference in its entirety.

Particular embodiments of the invention are directed to processes for producing a liquid hydrocarbon product (e.g., comprising naphtha boiling-range hydrocarbons, jet fuel boiling-range hydrocarbons, and/or diesel boiling-range hydrocarbons), as well as liquid hydrocarbon products obtained from such processes, including those internal to the process (e.g., present in a process stream, such as in an FT synthesis effluent or polishing effluent as described herein) or external to the process (e.g., recovered C₄⁺ hydrocarbon fractions as described herein). These include liquid hydrocarbon products in which at least a portion (e.g., at least about 70% on a weight or molar basis) of the carbon content of the hydrocarbons contained in these products is renewable carbon. Representative processes comprise a first stage for carrying out reforming and/or RWGS reactions, i.e., in a reforming stage, in an RWGS stage, or in a reforming/RWGS stage, on a gaseous feed mixture. This is followed by a second stage, namely a Fischer-Tropsch (FT) synthesis stage, of converting a synthesis gas intermediate produced in the first stage and comprising both H₂ and CO (i.e., an H₂/CO mixture). In particular, this intermediate is converted to C₄⁺ hydrocarbons contained in the liquid hydrocarbon product. The converting step is performed at least partially via FT synthesis, and optionally this step comprises a combination of both FT synthesis and wax cracking, optionally further in combination with isomerization, with the wax cracking reaction serving to reduce the molecular weight of hydrocarbons otherwise obtained from FT synthesis alone (in the absence of wax cracking), and preferably with the combination of wax cracking and isomerization reactions serving to dewax an FT synthesis effluent and/or a polishing effluent containing, or otherwise containing (in the absence of wax cracking and isomerization), a wax fraction comprising normal C₂₀⁺ hydrocarbons that are solid at room temperature. That is, wax cracking in combination with FT synthesis, and preferably wax cracking and isomerization in combination with FT synthesis, may serve to effectively reduce, or eliminate, the amount of such normal C₂₀⁺ hydrocarbons otherwise contained in the FT synthesis effluent and/or polishing effluent in the case of a polishing reactor being used in the FT synthesis stage. This is achieved, as described above, by converting a wax fraction (e.g., comprising normal C₂₀⁺ hydrocarbons) otherwise contained in an FT synthesis effluent (in the absence of cracking), to normal or branched C₄-C₁₉ hydrocarbons that contribute to the yield of liquid hydrocarbons from the process (e.g., the yield of such hydrocarbons present in recovered C₄⁺ hydrocarbon fractions as described herein).

Wax cracking, optionally in combination with isomerization, may be performed subsequent to FT synthesis, such as in a separate downstream wax cracking reactor, or otherwise may be performed simultaneously with FT synthesis, such as in the case of using, in an FT reactor, a mixture of an FT catalyst and a cracking catalyst, or otherwise a bi-functional FT/cracking catalyst having both an FT functional constituent and a cracking-functional constituent. In the case of either a separate wax cracking reactor or an FT reactor in which at least some wax cracking is performed, the effluent obtained from sequential or simultaneous wax cracking in the FT synthesis stage may be referred to as an FT synthesis effluent. According to more specific embodiments, the FT synthesis stage may comprise both (i) an FT reactor, for performing, simultaneously with FT synthesis, wax cracking, optionally in combination with isomerization (e.g., using a catalyst mixture or bi-functional catalyst), and (ii) a polishing reactor downstream of the FT reactor, for performing, separately from the FT synthesis, further wax cracking, optionally in combination with further isomerization. The polishing reactor may contain a one or more polishing catalysts (e.g., wherein at least one such polishing catalyst has the same composition and/or the same form as the cracking catalyst contained in the FT reactor, but being in the absence of the FT catalyst, or otherwise having the same composition as the cracking-functional constituent of the bi-functional catalyst contained in the FT reactor, such as having same composition as this bi-functional catalyst, but excluding the FT-functional constituent), such that the FT reactor may provide an FT synthesis effluent and the polishing reactor may provide a polishing effluent.

According to specific embodiments, in the first stage, a gaseous feed mixture comprising predominantly (i) H₂ and CO₂ or (ii) a hydrogen source and CO₂ is contacted with a catalyst as described herein (e.g., a reforming/RWGS catalyst) to produce the synthesis gas intermediate. Other particular embodiments are directed to processes described above, according to which biogas is converted to the liquid hydrocarbon product, i.e., the gaseous feed mixture is, or comprises, biogas. Advantageously, biogas provides a readily available gaseous feed mixture, or portion thereof, which comprises predominantly CH₄ and CO₂. Importantly, the carbon content of C₄⁺ hydrocarbons of liquid hydrocarbon products made in this manner is derived from CH₄ and CO₂ originating from organic waste, i.e., the carbon content is renewable. Other particular embodiments are directed to processes described above, according to which a gas derived from the decomposition, combustion, or gasification or biomass, optionally incorporating supplemental H₂, comprising CO, H₂, CO₂, and optionally CH₄, is converted to the liquid hydrocarbon product, i.e., the gaseous feed mixture is, or comprises, a gas derived from the decomposition, combustion, or gasification or biomass, optionally incorporating supplemental H₂. Representative processes according to these particular embodiments comprise, in a reforming stage (and possibly, but not necessarily, a reforming/RWGS stage), contacting a gas mixture comprising CO₂ in combination with H₂ and/or a hydrogen source, biogas (or a gaseous feed mixture comprising biogas), and/or a gas derived from the decomposition, combustion, or gasification or biomass, optionally incorporating supplemental H₂, comprising CO, H₂, CO₂, and optionally CH₄, with a reforming/RWGS catalyst to produce a synthesis gas intermediate comprising an H2/CO mixture. The processes may further comprise, in an FT synthesis stage, converting the synthesis gas intermediate to the liquid hydrocarbon product, at least partially via FT synthesis and possibly a combination of both FT synthesis and wax cracking, optionally further in combination with isomerization, as described above.

Further aspects relate to the ability to recycle a fraction of the FT synthesis effluent or polishing effluent obtained from the FT synthesis stage, whether or not, in the case of the FT synthesis effluent, this effluent is obtained following optional wax cracking and/or optional isomerization. In particular, together with the liquid hydrocarbon product, the FT synthesis effluent or polishing effluent may comprise a fraction enriched in (i) H₂ and CO₂ or (ii) a hydrogen source and CO₂, which components (i) or (ii) of this fraction may include unconverted species and/or light hydrocarbon byproducts (e.g., CH₄, C₂H₆, C₃H₈) exiting the FT synthesis stage (e.g., an FT reactor used in this stage or a polishing reactor used in this stage). Representative processes may further comprise separating, from the FT synthesis effluent (whether or not obtained following optional wax cracking and/or optional isomerization) or the polishing effluent, (A) the liquid hydrocarbon product comprising the C₄⁺ hydrocarbons, and (B) the fraction enriched in (i) H₂ and CO₂ or (ii) a hydrogen source and CO₂. The fraction (B) may be recycled to the reforming stage, the RWGS stage, or the reforming/RWGS stage, or may be recycled to the FT synthesis stage. Otherwise, portions of the fraction (B) may be recycled to these respective stages. The ability to operate with recycle in this manner resides in the effectiveness of reforming/RWGS catalysts described herein to produce H₂ and CO in the synthesis gas intermediate from (i) CO₂, together with H₂ (via the RWGS reaction), as well as from (ii) CO₂, together with a hydrogen source (via the dry reforming reaction). For example, in the case of a hydrogen source comprising light alkane hydrocarbons (e.g., one or more of CH₄, C₂H₆, C₃H₈), the dry reforming reaction can proceed according to the following general reaction:

CₙH₂ₙ₊₂+nCO₂→2CO+(n+1)H₂.

Operation with recycle of a fraction of the FT synthesis effluent or of the polishing effluent, obtained from separation of the liquid hydrocarbon fraction from such effluent, can advantageously provide high overall conversion of input carbon, including CO₂ (or the carbon present in CO₂, in combination with carbon from any hydrocarbons and any other carbon sources) approaching 100% as all of the reactants of the RWGS and dry reforming reactions, including CO₂, are recycled to extinction, as well as a high overall utilization of input carbon, including CO₂, in the formation of C₄⁺ hydrocarbons as described herein. The fraction recycled may be enriched, relative to the FT synthesis effluent or polishing effluent, and also relative to the synthesis gas intermediate, in (i) H₂ and CO₂, based on their combined amount in the recycled fraction, or (ii) a hydrogen source and CO₂, based on their combined amount in the recycled fraction, and is generally enriched in both (i) and (ii). In this regard, important advantages associated with the present invention relate to the ability of reforming/RWGS catalysts described herein to process a “light ends” fraction of the FT synthesis effluent or of the polishing effluent, enriched in (i) or (ii), which is recycled to the first stage of the process, thereby ultimately converting up to the stoichiometric limits substantially all, or all, of the carbon fed to the process, including CO₂, (e.g., in a fresh makeup feed or, more particularly, a fresh makeup CO₂- and/or CH₄-containing feed) to C₄⁺ hydrocarbons. The composition of the input feed may be preferentially controlled to yield a feed having the right stoichiometry for substantially all, or all, of the carbon fed to the process, including CO₂, to be converted to C₄⁺ hydrocarbons. This provides superior yields of liquid hydrocarbons, based on CO₂ carbon that is fed or input to the process, compared to processes in which the light ends fraction, or a substantial portion thereof, is not recycled. In the case of operation with recycle, for example, at least about 80%, at least about 90%, or even at least about 95%, of the CO₂ carbon that is fed to the process is converted to C₄⁺ hydrocarbons in the liquid hydrocarbon product and/or in separated and optionally recovered C₄⁺ hydrocarbon fractions. In the case of operation with recycle, for example, at least about 80%, at least about 90%, or even at least about 95%, of the input carbon that is fed to the process is converted to C₄⁺ hydrocarbons in the liquid hydrocarbon product and/or in separated and optionally recovered C₄⁺ hydrocarbon fractions.

In yet other representative processes utilizing recycle, all or a portion of a separated fraction of C₄⁺ hydrocarbons may be recycled to the reforming stage or RWGS stage to improve control of the product slate of recovered C₄⁺ hydrocarbons, output from the process. For example, one or more separated fractions of the process may include a naphtha boiling-range hydrocarbon fraction. In the case of recycling all or a portion of such fraction to the first stage of the process for reforming of the naphtha boiling-range hydrocarbons to provide additional synthesis gas, the product slate, or product yield of the process, may be shifted toward recovery of other types of hydrocarbons, such as jet fuel boiling-range hydrocarbons and/or diesel boiling-range hydrocarbons obtained in respective, separated and recovered fractions enriched in these hydrocarbons. The control of the product slate, or product yield, in this manner, is made possible by the flexibility of the reforming/RWGS catalyst, in terms of reforming a wide variety of hydrocarbons, in addition to CH4.

Overall, processes are described herein for producing liquid hydrocarbon products from undesirable CO₂, including waste gases containing CO₂. In some cases, if the CO₂ is extracted from air, the processes effectively reverse CO₂ emissions from the combustion of hydrocarbons, by restoring atmospheric CO₂ back to hydrocarbons. Further environmental benefits are realized in embodiments in which H₂ present in a gaseous feed mixture is produced from electrolysis of water, and particularly using electricity from renewal sources such as solar and wind energy, fossil hydrogen with CCS, bio-gasification hydrogen, or methane pyrolysis hydrogen. As can be appreciated by those skilled in the art having knowledge of the present disclosure, processes described herein have the potential to replace liquid hydrocarbon products conventionally refined from fossil fuels, including gasoline and diesel fuel, with renewable hydrocarbon products made from recycled CO₂ removed from the atmosphere. These processes can likewise convert CO₂ and renewable hydrocarbons, such as those in biogas or sourced from biomass generally (e.g., from gasification or biomass hydropyrolysis).

These and other embodiments, aspects, and advantages relating to the present invention are apparent from the following Detailed Description.

### BRIEF DESCRIPTION OF THE DRAWINGS

A more complete understanding of the exemplary embodiments of the present invention and the advantages thereof may be acquired by referring to the following description in consideration of the accompanying figures, providing flow schemes of processes for producing liquid hydrocarbon products, in which the same reference numbers are used to identify the same or similar features.

FIG. 1 depicts a flowscheme illustrating an embodiment of a process for producing a liquid hydrocarbon product comprising C₄⁺ hydrocarbons, utilizing two reaction stages, namely a reforming or RWGS stage and an FT synthesis stage, in combination with a separation stage. Advantageously, light ends such as unconverted CO and H₂, as well as CO₂ and optionally light hydrocarbons (e.g., CH₄, C2H6), separated downstream of the FT synthesis stage, can be recycled. In the particular embodiment of FIG. 2, the FT synthesis stage includes both an FT reactor and a polishing reactor.

FIG. 2 depicts a flowscheme illustrating an embodiment of a process for producing a liquid hydrocarbon product comprising C₄⁺ hydrocarbons, in which the FT synthesis stage more particularly includes both an FT reactor and a polishing reactor, with a portion of a fresh makeup H₂-containing feed optionally being fed directly to the polishing reactor. This figure also illustrates, in addition to the recycle of light ends as shown in FIG. 1, the optional recycle of at least a portion of a separated C₄⁺ hydrocarbon fraction, such as a naphtha boiling-range hydrocarbon fraction.

FIG. 3 depicts a distillation curve obtained for a liquid hydrocarbon product recovered from FT synthesis, with demarcations of the gasoline, jet fuel, and diesel boiling-range hydrocarbons.

FIG. 4 depicts the performance of a reforming catalyst, also having activity for catalyzing the reverse water-gas shift (RWGS) reaction, in terms of total hydrocarbon conversion, over an operating period in excess of 500 hours.

FIG. 5 depicts the performance of the reforming catalyst used to obtain the results shown in FIG. 4, but in terms of the H₂:CO molar ratio of synthesis gas produced over the operating period.

FIG. 6 depicts the performance of the reforming catalyst used to obtain the results shown in FIGS. 4 and 5, but in terms of the reformer product composition over the operating period.

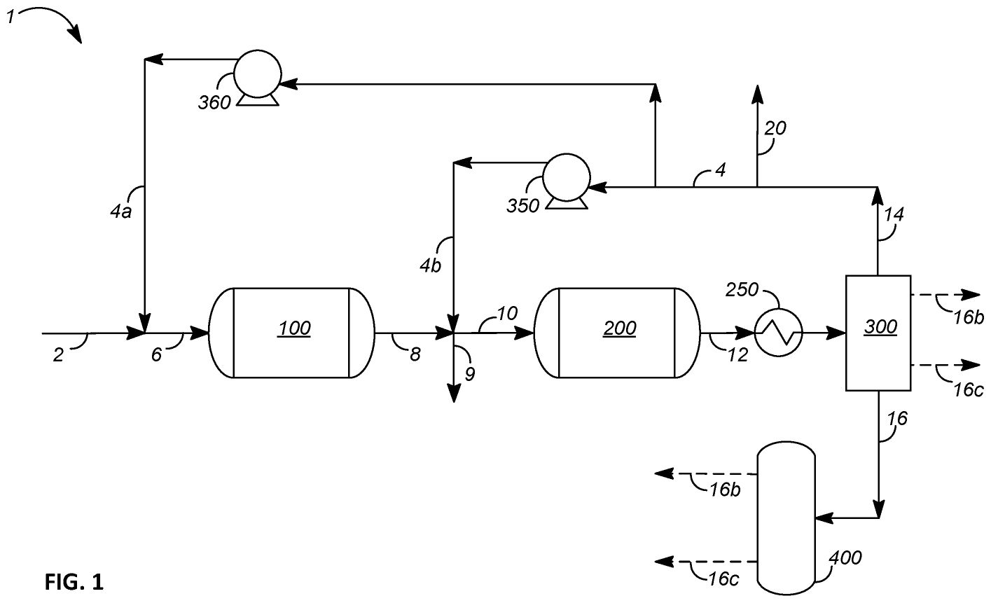

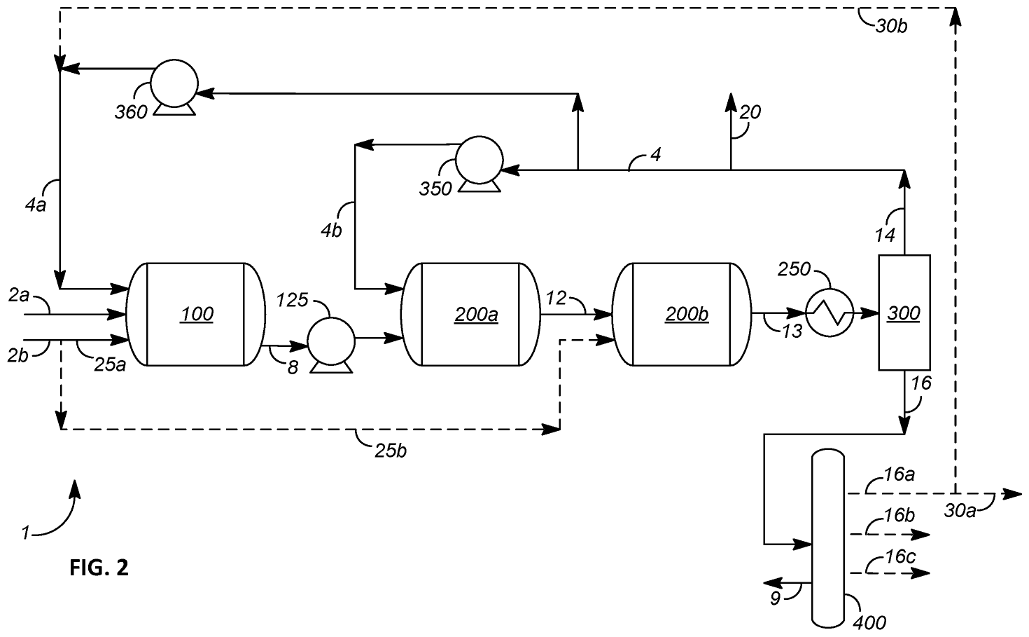

FIGS. 1 and 2 should be understood to present illustrations of processes and certain principles involved. In order to facilitate explanation and understanding, these figures provide a simplified overview, with the understanding that the depicted elements are not necessarily drawn to scale. The processes illustrated in FIGS. 1 and 2 illustrate a number of possible features as described herein, which features may be implemented individually or in any combination. That is, not all features (e.g., not all individual operations and their associated process streams and equipment) are required in, or essential to, the practice of various inventive embodiments described herein, i.e., it should be understood that various specific features can be implemented independently of others. In order to further facilitate explanation and understanding, FIGS. 1 and 2 provide an overview of various features for implementation in reforming in combination with FT synthesis. Some associated equipment such as certain vessels, heat exchangers, valves, instrumentation, and utilities, are not shown, as their specific description is not essential with respect to the understanding or practice of various inventive embodiments. Such equipment would be readily apparent to those skilled in the art, having knowledge of the present disclosure. Other processes for producing liquid hydrocarbon products, such as renewable fuels, via the reactions of reforming and/or RWGS according to other embodiments within the scope of the invention and having configurations and constituents determined, in part, according to particular processing objectives, would likewise be apparent.

### DETAILED DESCRIPTION

The expressions “wt-%” and “mol-%,” are used herein to designate weight percentages and molar percentages, respectively. The expressions “wt-ppm” and “mol-ppm” designate weight and molar parts per million, respectively. For ideal gases, “mol-%” and “mol-ppm” are equal to percentages by volume and parts per million by volume, respectively. In some cases, a percentage, “%,” is given with respect to values that are the same, whether expressed as a weight percentage or a molar percentage. For example, (i) the percentage of the feed carbon content that forms C₄⁺ hydrocarbons of the liquid hydrocarbon product, or (ii) the percentage of the carbon content of the liquid hydrocarbon product that is renewable carbon or carbon derived from CO₂, has the same value, whether expressed as a weight percentage or a molar percentage.

The terms “substantially” and “substantial,” as used herein, refer to an extent of at least 95 mol-% in the case of gases being referred to, and at least 95 wt-% in the case of liquids or solids being referred to. For example, the phrase “substantially all” may be replaced by “at least 95 mol-%” or “at least 95 wt-%,” as the case may be. With respect to a referenced item being “substantially absent” or having a “substantial absence,” this should be understood to mean that the item is present in an amount of at most 5 mol-%, or of at most 5 wt-%, as the case may be, of the referenced total. In preferred embodiments, “substantially all” may be replaced by “all,” “substantially absent” may be replaced by “absent,” and “substantial absence” may be replaced by “absence.”

The term “liquid hydrocarbon product” refers to a product that comprises hydrocarbons that are liquid at room temperature. Examples of these are hydrocarbons having 4 or more carbon atoms, i.e., “C₄⁺ hydrocarbons” as referred to herein.

“A hydrogen source” refers to one or more compounds that can generate hydrogen according to

various reactions described herein and occurring in the reforming stage, the RWGS stage, or the reforming/RWGS stage. Examples of such compounds are CH₄, C₂H₆, C3H8, and H₂O, and a hydrogen source may comprise one or more of these compounds. According to specific embodiments, a hydrogen source may comprise one or more of CH₄, C₂H₆, C₃H₈ that generate hydrogen according to the dry reforming reaction as described herein. According to specific embodiments, a hydrogen source may comprise one or more of CH₄, C₂H₆, C₃H₈ that generate hydrogen according to the steam reforming reaction as described herein. For example, with respect to any embodiment described herein, “a hydrogen source” may refer to CH₄, and amounts (e.g., concentrations) of a hydrogen source may refer to methane alone. Alternatively, “a hydrogen source” may refer to CH₄ and C2H6 in combination, and amounts (e.g., concentrations) of a hydrogen source may refer to a combined amount of methane and ethane. Alternatively, “a hydrogen source” may refer to CH₄, C₂H₆, and C₃H₈ in combination, and amounts (e.g., concentrations) of a hydrogen source may refer to a combined amount of methane, ethane, and propane. Alternatively, “a hydrogen source” may refer to CH₄, C₂H₆, C₃H₈,and H₂O in combination, and amounts (e.g., concentrations) of a hydrogen source may refer to a combined amount of methane, ethane, propane, and water (e.g., in the form of steam).

The term “naphtha boiling-range hydrocarbons,” which is synonymous with “gasoline boiling-range hydrocarbons” and which may be replaced by this term, refers to a hydrocarbon fraction comprising hydrocarbons having boiling points within an initial (“front-end”) distillation temperature characteristic of Cs hydrocarbons, for example from about 30° C. (86° F.) to about (104° F.), with a representative value being 35° C. (95° F.) and an end point distillation temperature generally from about 130° C. (266° F.) to about 169° C. (336° F.), and typically from about 141° C. (286° F.) to about 163° C. (325° F.), with a representative value being 155° C. (311° F.). The terms “jet fuel boiling-range hydrocarbons” and “diesel boiling-range hydrocarbons” refer to a hydrocarbon fractions comprising hydrocarbons having boiling points within a front-end distillation temperature from about 135° C. (275° F.) to about 175° C. (347° F.), with a representative value being 155° C. (311° F.). The distillation end point temperature of jet fuel boiling-range hydrocarbons is generally from about 275° C. (527° F.) to about 300° C. (572° F.), with a representative value being 285° C. (545° F.), whereas the distillation end point temperature of diesel fuel boiling-range hydrocarbons is generally from about 300° C. (572° F.) to about 400° C. (752° F.)), with a representative value being 370° C. (698° F.). These boiling point temperatures, which are also characteristic of respective petroleum derived gasoline, jet fuel, and diesel boiling-range fractions, may be measured according to ASTM D86, with the end point being the 95% recovery value. For purposes of characterizing (i) naphtha boiling-range hydrocarbons, (ii) jet fuel boiling-range hydrocarbons, and (iii) diesel boiling-range hydrocarbons, according to some embodiments, these may be considered, respectively, hydrocarbon fractions comprising hydrocarbons having normal boiling points (i) between 35° C. (95° F.) and 135° C. (275° F.), (ii) between 135° C. (275° F.) and 300° C. (572° F.), and (iii) between 300° C. (572° F.) and 400° C. (752° F.). Such fractions may be readily ascertained, for example, from fractionation of a liquid hydrocarbon product obtained from processes described herein (e.g., following its separation from an H₂/CO₂-enriched fraction or a hydrocarbon/CO₂-enriched fraction).

In representative processes described herein, a first or initial stage may be referred to as “a reforming/RWGS stage” to indicate that both reforming and reverse water-gas shift (RWGS) reactions occur to some extent. Reforming, as understood in the art and in the context of the present disclosure, refers to the reaction of CH₄ and/or possibly other hydrocarbons (e.g., those hydrocarbons that contribute to a hydrogen source as described above, such as C₂H₆ and/or C₃H₈) with an oxidant to produce H₂ and CO (synthesis gas), with the oxidant preferably including CO₂, but possibly comprising any one or more of CO₂, H₂O, and O₂.The RWGS reaction is understood in the art as the following:

H₂+CO₂→H₂O+CO.

In broader embodiments, the first or initial stage may be “a reforming stage,” in which the reforming of CH₄ and/or possibly other hydrocarbons occurs as noted above, whereas the RWGS reaction does not necessarily occur. In other broader embodiments, the first or initial stage may be “an RWGS stage,” in which the RWGS reaction occurs as noted above, whereas the reforming of CH₄ and/or possibly other hydrocarbons does not necessarily occur. For example, in the case of a gaseous feed mixture comprising CH₄ and CO₂, the first stage may be a reforming stage in which these components react to produce synthesis gas. Typically, however, at least some H₂ of the synthesis gas, and present in the reaction mixture, reacts with CO₂, also present in the reaction mixture, according to the RWGS reaction, such that the reforming stage may be more specifically characterized as “a reforming/RWGS stage.” In the case of a gaseous feed mixture comprising H₂ and CO₂, the first stage may be an RWGS stage in which these components react as noted above. It can be appreciated, therefore, that the first or initial stage may be either a reforming stage or an RWGS stage, in the case of a gaseous feed mixture comprising, together with CO₂, either CH₄ (and/or possibly other hydrocarbons such as C₂H₆ and/or C₃H₈) or H₂, respectively. In the case of any gaseous feed mixture comprising CH₄ (and/or possibly other hydrocarbons such as C₂H₆ and/or C₃H₈) together with CO₂ (e.g., comprising CH₄, CO₂, and H₂) the first or initial stage may be a reforming/RWGS stage.

### Gaseous Feed Mixtures

Exemplary processes, for producing a liquid hydrocarbon product comprising C₄⁺ hydrocarbons, include (a) in a reforming stage or an RWGS stage, contacting a gaseous feed mixture with a reforming/RWGS catalyst to produce a synthesis gas intermediate comprising an H₂/CO mixture; and (b) converting the synthesis gas intermediate to the liquid hydrocarbon product, at least partially via Fischer-Tropsch (FT) synthesis. Representative gaseous feed mixtures comprise predominantly (i) H₂ and CO₂ or (ii) a hydrogen source and CO₂, with the term “predominantly” referring to these gaseous feed mixtures comprising (i) H₂ and CO₂ in a combined amount of at least 50 mol-%, or (ii) a hydrogen source (e.g., which may comprise one or more compounds as described above) and CO₂ in a combined amount of at least 50 mol-%. In more specific embodiments, gaseous feed mixtures comprise (i) H₂ and CO₂ in a combined amount of at least 75 mol-%, at least about 90 mol-%, or at least about 95 mol-%, or (ii) a hydrogen source and CO₂ in a combined amount of at least 75 mol-%, at least about 90 mol-%, or at least about 95 mol-%. According to other embodiments, representative gaseous feed mixtures may comprise CH₄, CO₂, and H₂ in a combined amount of at least 50 mol-%, at least about 75 mol-%, at least about 90 mol-%, or at least about 95 mol-%. According to other embodiments, representative gaseous feed mixtures may comprise CO, CO₂, and H₂ in a combined amount of at least 50 mol-%, at least about 75 mol-%, at least about 90 mol-%, or at least about 95 mol-%. Alternatively, or in combination with any of the features described herein, representative gaseous feed mixtures may comprise little or no amounts of other components. For example, in the case of a gaseous feed mixture comprising predominantly (i) H₂ and CO₂, such gaseous feed mixture may comprise a hydrogen source (e.g., may comprise CH₄, optionally in combination with other hydrocarbons such as C₂H₆ and/or C₃H₈) in an amount of less than about 25 mol-%, less than about 10 mol-%, less than about 5 mol-%, or less than about 1 mol-%. In the case of a gaseous mixture comprising predominantly (ii) a hydrogen source (e.g., which may comprise one or more compounds as described above) and CO₂, such gaseous feed mixture may comprise H₂ in an amount of less than about 25 mol-%, less than about 10 mol-%, less than about 5 mol-%, or less than about 1 mol-%. In the case of a gaseous feed mixture comprising predominantly (i) H₂, CO₂ and CO, such gaseous feed mixture may comprise a hydrogen source (e.g., may comprise CH₄, optionally in combination with other hydrocarbons such as C₂H₆ and/or C₃H₈) in an amount of less than about 25 mol-%, less than about 10 mol-%, less than about 5 mol-%, or less than about 1 mol-%. Any gaseous feed mixture described herein may comprise oxygen-containing components other than CO₂, for example, one or more of CO, H₂O, and 02 in a respective amount (individually), or in a combined amount, of less than, about 35 mol-%, about 15 mol-%, about 10 mol-%, less than about 5 mol-%, or less than about 1 mol-%. In such cases, due to the limited presence, or absence, of oxidants other than CO₂, any reforming of CH₄ that occurs in a reforming stage or in a reforming/RWGS stage may be substantially, or entirely, dry reforming and/or may be substantially, or entirely, unaccompanied by partial oxidation.

In the case of the gaseous feed mixture comprising predominantly (ii) a hydrogen source (e.g., CH₄ alone or optionally in combination with other hydrocarbons such as C₂H₆ and/or C₃H₈) and CO₂, step (a) may be a reforming stage, and optionally a reforming/RWGS stage, as described above, according to which, in either case, H₂ and CO in the H₂/CO mixture of the synthesis gas intermediate may be produced from the reaction of CH₄ and CO₂. In the case of the gaseous feed mixture comprising predominantly (i) H₂ and CO₂, step (a) may be an RWGS stage, and optionally a reforming/RWGS stage, as described above. If step (a) is an RWGS stage, H₂ in the H₂/CO mixture of the synthesis gas intermediate may be H₂ that is unreacted, or that represents an equilibrium amount, in the RWGS reaction of H₂ and CO₂ as described above, whereas CO in this H₂/CO mixture may be CO that is produced in the RWGS reaction or alternatively may be CO that is unreacted. If step (a) is a reforming/RWGS stage, the gaseous feed mixture comprising predominantly (i) H₂ and CO₂ may further comprise CH₄, optionally in combination with other hydrocarbons such as C₂H₆ and/or C3H8. Therefore, H₂ and CO in the H₂/CO mixture of the synthesis gas intermediate may be produced from the reaction of CH₄ (optionally in combination with other hydrocarbons such as C₂H₆ and/or C₃H₈) and CO₂. It may be further appreciated that, whether or not the gaseous feed mixture comprises CH₄ (optionally in combination with other hydrocarbons such as C₂H₆ and/or C₃H₈) such that H₂ may be produced from reforming, the H₂ and CO in the H₂/CO mixture of the synthesis gas intermediate may represent equilibrium amounts in the RWGS reaction. In specific embodiments in which the gaseous feed mixture comprises CH₄ (optionally in combination with other hydrocarbons such as C₂H₆ and/or C₃H₈), the H₂ and CO in the H₂/CO mixture of the synthesis gas intermediate may represent equilibrium amounts in combined reforming and RWGS reactions. To the extent that, in a reforming stage or reforming/RWGS stage, CH₄ (optionally in combination with other hydrocarbons such as C₂H₆ and/or C₃H₈) and CO₂ are reacted according to the dry reforming reaction described above, the reaction of CH₄ (optionally in combination with other hydrocarbons such as C₂H₆ and/or C₃H₈) with one or both of the other oxidants H₂O and O₂ may also produce H₂ and/or CO in the H₂/CO mixture of the synthesis gas intermediate. For example, these other oxidants may also be present in the gaseous feed mixture, or, alternatively, H₂O may be present in the reaction mixture (although not necessarily present in the gaseous feed mixture) as a product of the RWGS reaction.

The gaseous feed mixture, or at least the compounds present in this mixture (e.g., CO₂, CH₄, and/or H₂), may be obtained from a wide variety of sources. Advantageously, such sources include waste gases that are regarded as having little or no economic value or gases derives from wastes that are regarded as having little or no economic value, and that may otherwise contribute to atmospheric CO₂ levels. For example, the gaseous feed mixture may be, or may comprise, an industrial process waste gas that is obtained from a steel manufacturing process or a non-ferrous product manufacturing process. Other processes from which all or a portion of the gaseous feed mixture may be obtained include petroleum refining processes (e.g., processes producing refinery off gases), renewable hydrocarbon fuel (biofuel) production processes (e.g., pyrolysis processes, such as hydropyrolysis processes, or a fatty acid/triglyceride hydroconversion processes), biomass and coal (e.g., lignocellulose and char) gasification processes, electric power production processes, carbon black production processes, ammonia production processes, other chemical (e.g., methanol) production processes, and coke manufacturing processes. In some cases, the gaseous feed mixture may be, or may comprise, (i) a wellhead gas comprising methane or (ii) a gaseous product of the electrochemical reduction of carbon dioxide.

According to some embodiments, the gaseous feed mixture may comprise CO₂ obtained from direct air capture (DAC) (i.e., CO₂ extracted from the atmosphere). Alternatively, or in combination, the gaseous feed mixture may comprise H₂ obtained from the electrolysis of water, such as by the use of renewable electricity (e.g., generated from wind or solar energy). For example, the gaseous feed mixture may comprise CO₂ and H₂ (e.g., in a combined amount as described above), with all or substantially all the CO₂ being obtained from direct air capture and/or all or substantially all of the H₂ being electrolysis hydrogen (i.e., hydrogen obtained from the electrolysis of water.) Alternatively, such hydrogen may be fossil hydrogen with carbon capture and sequestration (CCS), bio-gasification hydrogen, or methane pyrolysis hydrogen.

A particular gaseous feed mixture of interest is biogas, which is understood to include products of anaerobic bacterial digestion of biowastes as well as landfill gases. Typically, biogas contains methane in an amount from about 35 mol-% to about 90 mol-% (e.g., about 40 mol-% to about 80 mol-% or about 50 mol-% to about 75 mol-%) and CO₂ in an amount from about 10 mol-% to about 60 mol-% (e.g., about 15 mol-% to about 55 mol-% or about 25 mol-% to about 50 mol-%). The gases N₂, H₂, H₂S, and O₂ may be present in minor amounts (e.g., in a combined amount of less than 20 mol-%, or less than 10 mol-%). In some embodiments, therefore, a gaseous feed mixture may be, or may comprise, biogas.

Another gaseous feed mixture of interest is natural gas comprising methane in an amount from about 65 mol-% to about 98 mol-% (e.g., about 70 mol-% to about 95 mol-% or about 75 mol-% to about 90 mol-%) and CO₂ in an amount from about 3 mol-% to about 35 mol-% (e.g., about 5 mol-% to about 30 mol-% or about 10 mol-% to about 25 mol-%). Other hydrocarbons (e.g., ethane and propane), as well as nitrogen, may be present in minor amounts. Of particular interest is stranded natural gas, which, using known processes, is not easily converted to a synthesis gas intermediate in an economical manner. In some embodiments, therefore, a gaseous feed mixture may be, or may comprise, natural gas, for example comprising a relatively high amount of CO₂, such as at least about 10 mol-% or even at least about 25 mol-%.

A further gaseous feed mixture of interest is a hydrogen-depleted PSA tail gas, for example obtained from a hydrogen production processes involving steam methane reforming (SMR), as described above. This mixture may comprise (i) methane in an amount from about 5 mol-% to about 45 mol-% (e.g., about 10 mol-% to about 35 mol-% or about 15 mol-% to about 25 mol-%), (ii) CO₂ in an amount from about 20 mol-% to about 75 mol-% (e.g., about 25 mol-% to about 70 mol-% or about 35 mol-% to about 60 mol-%), and (iii) an H₂ in an amount from about mol-% to about 45 mol-% (e.g., about 15 mol-% to about 40 mol-% or about 20 mol-% to about 35 mol-%). The balance of this stream may comprise predominantly water vapor and/or CO. In some embodiments, therefore, a gaseous feed mixture may be, or may comprise, a hydrogen-depleted PSA tail gas.

A further gaseous feed mixture of interest is a gaseous effluent from a biological (bacterial) fermentation that is integrated with a hydrogen production process. Such integrated fermentation processes are described, for example, in U.S. Pat. Nos. 9,605,286; 9,145,300; US 2013/0210096; and US 2014/0028598. Such gaseous effluent may comprise (i) methane in an amount from about 5 mol-% to about 55 mol-% (e.g., about 5 mol-% to about 45 mol-% or about 10 mol-% to about mol-%), (ii) CO₂ in an amount about 5 mol-% to about 75 mol-% (e.g., about 5 mol-% to about 60 mol-% or about 10 mol-% to about 50 mol-%), and (iii) an H₂ in an amount from about mol-% to about 40 mol-% (e.g., about 5 mol-% to about 30 mol-% or about 10 mol-% to about mol-%). The balance of this stream may comprise predominantly water vapor and/or CO. In some embodiments, therefore, a gaseous feed mixture may be, or may comprise, such gaseous effluent from fermentation.

A further gaseous feed mixture of interest is a gaseous effluent from biomass gasification, optionally supplemented with additional H₂. Such gaseous effluent may comprise (i) methane in an amount from about 0 mol-% to about 15 mol-% (e.g., about 0 mol-% to about 10 mol-% or about 0 mol-% to about 5 mol-%), (ii) CO₂ in an amount about 0 mol-% to about 60 mol-% (e.g., about 5 mol-% to about 50 mol-% or about 15 mol-% to about 45 mol-%), and (iii) an H₂ in an amount from about 5 mol-% to about 40 mol-% (e.g., about 5 mol-% to about 30 mol-% or about 10 mol-% to about 25 mol-%). The balance of this stream may comprise predominantly water vapor and/or CO. In some embodiments, therefore, a gaseous feed mixture may be, or may comprise, such gaseous effluent from biomass gasification, optionally supplemented with additional H₂.

In some embodiments, the compositions of gaseous feed mixtures as described herein may be representative of a combined composition of two or more streams being separately fed to a reactor used in the reforming stage or the RWGS stage. Separate streams may include, for example, recycle streams or streams of one species, or enriched in one species (e.g., a CH₄-enriched stream), relative to the gaseous feed mixture. In particular embodiments according to which recycle is utilized, the gaseous feed mixture may be provided to such reactor or reaction stage as a combination of (A) a fresh makeup feed and (B) a fraction enriched in (i) H₂ and CO₂ or (ii) the hydrogen source and CO₂, as described herein. That is, the gaseous feed mixture may comprise (A) and (B), such that a fresh makeup feed may, according to particular embodiments associated with any “gaseous feed mixture” described herein, be a portion of such gaseous feed mixture. Components of the gaseous feed mixture may therefore include (A) fresh gaseous feed mixture components, which namely serve as inputs to the overall process, and (B) recycle gaseous feed mixture components. Particular examples of (A) include a fresh makeup CO₂-and/or CH₄-containing feed and a fresh makeup H₂-containing feed, the former of which may include CO₂ obtained from DAC, the exhaust associated with the combustion of biomass, or biomass gasification, and the latter of which may include H₂ that is electrolysis hydrogen, fossil hydrogen with carbon capture and sequestration (CCS), bio-gasification hydrogen, or methane pyrolysis hydrogen. An exemplary fresh makeup CO₂- and/or CH₄-containing feed includes sustainable carbon, such as from biogenic sources, and may therefore include (a) CO₂ obtained from DAC, (b) CO₂ and/or CH₄ obtained from biogas, and/or (c) CO₂, CO, and/or CH₄ obtained from biomass gasification. Particular examples of (B) include a fraction enriched in (i) H₂ and CO₂ or (ii) the hydrogen source and CO₂, as well as a hydrocarbon recycle, namely a recycle of at least a portion of a separated fraction enriched in C₄⁺ hydrocarbons, such as a fraction enriched in naphtha boiling-range hydrocarbons.

To the extent that any components (A) and (B) of the gaseous mixture may be combined prior to (upstream of) a reactor used in the reforming stage or the RWGS stage, or may otherwise be added directly to this reactor in separate streams, it can be appreciated that the “gaseous feed mixture” may refer, in certain embodiments, to the composition formed within this reactor (e.g., in situ, such as at the reactor inlet) or at least the composition that is represented by combining components (A) and (B). With respect to the various fresh and recycle components of the gaseous feed mixture, and considering the various possible combinations of such components, in general the gaseous feed mixture that is fed to a reactor used in the reforming stage or the RWGS stage may comprise CO₂, CO, H₂, and light ends (e.g., CH₄ and optionally other light hydrocarbons such as C₂H₆ and C₃H₈), with supplemental H₂O (steam) being added as needed to promote SMR and possibly tailor the H₂:CO molar ratio of the synthesis gas intermediate. To the extent that (A) fresh gaseous feed mixture components may include light hydrocarbons (e.g., predominantly CH₄), in combination with CO₂ and H₂, and that H₂O (steam) may also be present in the gaseous feed mixture, for example due to the RWGS reaction and/or due to (B) recycle gaseous feed mixture components, a reforming/RWGS reactor used in the first stage of the process may be considered a “Tri-Converting” reactor, insofar as this reactor is used to carry out reactions of (i) dry reforming, (ii) steam reforming, and (iii) RWGS, as needed to produce the synthesis gas intermediate comprising an H₂:CO mixture.

Any description of (i) a fraction enriched in H₂ and CO₂ or (ii) a fraction enriched in the hydrogen source and CO₂, can, according to alternative embodiments, refer more specifically to “a portion” of such fraction (i) or (ii), for example a recycle portion of this fraction, or even a part of such recycle portion, consistent with the further disclosure below, including reference to FIGS. 1 and 2. For example, a purge stream, sampling streams, etc. may be removed from a fraction of the FT synthesis effluent that is enriched in (i) H₂ and CO₂ or (ii) the hydrogen source and CO₂, leaving only a recycle portion of such fraction (i) or (ii) to be returned to the process, such as to the first stage (e.g., a reforming stage, such as a reforming/RWGS stage, or an RWGS stage) and/or the FT synthesis stage, optionally with different parts of such recycle portion of fraction (i) or (ii) being routed to different stages. In the same manner, any description of a hydrocarbon recycle, such as a recycle of a separated fraction enriched in C₄⁺ hydrocarbons, can refer more specifically to a portion of such fraction. In view of the above description, and further description herein relating to recycle operation, the gaseous feed mixture may comprise a fresh makeup feed, optionally in combination with a recycle portion of (i) a fraction enriched in H₂ and CO₂ or (ii) a fraction enriched in the hydrogen source and CO₂ (or even a part of such fraction (i) or (ii)) that is separated from an FT synthesis effluent, and/or optionally in combination with a hydrocarbon recycle.

According to the above description, in some embodiments, compositions of gaseous feed mixtures as described herein may be representative of a combined composition of two or more streams being separately fed, or input, to a reactor used in the reforming stage or the RWGS stage. Separate streams may include, for example, fresh feed and/or recycle streams (e.g., a fresh makeup feed and/or (A) a fraction (i) or (ii) as described herein, or a recycle portion of such fraction and/or (B) a hydrocarbon recycle) or streams of one component, or enriched in one component (e.g., a CH₄-enriched stream), relative to the gaseous feed mixture. Any of the composition features described above with respect to a gaseous feed mixture can, according to alternative embodiments, apply to a fresh makeup feed that may be, for example, a portion of the gaseous feed mixture that is fed, or input, to a reactor used in the reforming stage or the RWGS stage, such as in the case of recycle operation.

### Reforming/RWGS Catalysts

As described above, an important aspect associated with the invention is the discovery that catalysts described herein can catalyze both the reforming (including dry reforming) of hydrocarbons (e.g., CH₄, C₂H₆, and/or C₃H₈) and the RWGS reaction, to various extents that depend on the composition of the particular gaseous feed mixture, as described above, and particular reforming/RWGS conditions used. This provides considerable flexibility with respect to compositions of gaseous feed mixtures that may be processed into a synthesis gas intermediate using reforming and/or RWGS reactions. As used herein, the term “reforming/RWGS catalyst” refers to a catalyst having at least some activity for catalyzing reforming and/or at least some activity for catalyzing RWGS in an initial stage of the process, whether such stage may be characterized as a reforming stage or an RWGS stage. In preferred embodiments, such catalyst will catalyze both reactions to at least some extent, in a reforming/RWGS stage, given the gaseous feed mixture and conditions used.

Representative embodiments comprise contacting, in a reforming stage or an RWGS stage, a gaseous feed mixture as described herein with a reforming/RWGS catalyst. This contacting may be performed batchwise, but preferably is performed continuously, with a continuous flow of the gaseous feed mixture to one or more reactors (and preferably to a single reactor) used in this stage that contain the reforming/RWGS catalyst (e.g., such that this catalyst is disposed in a catalyst bed volume within the reactor). The reforming stage or the RWGS stage may therefore likewise include the continuous withdrawal from the reactor(s) of the synthesis gas intermediate comprising an H₂/CO mixture, i.e., the intermediate product comprising both H₂ and CO, wherein such H₂ and CO may be unreacted gases (present in the gaseous feed mixture) or may be produced from reforming and/or RWGS reactions as described above.

Catalysts described herein exhibit a number of important advantages compared to conventional reforming catalysts, particularly in terms of tolerance to certain components that may be present in the gaseous feed mixture, such as C₂⁺ olefinic hydrocarbons and/or H₂S or other sulfur-bearing components (e.g., mercaptans). Such characteristics reduce the significant pretreating requirements of conventional processes and thereby improve flexibility, in terms of economically producing the synthesis gas intermediate, even on a relatively small operating scale, from common process streams containing significant concentrations of such components. In some embodiments, any of the gaseous feed mixtures described herein may comprise, in addition to CO₂, CH₄, and/or H₂, one or more C₂⁺ olefinic hydrocarbons, such as ethylene, propylene, butene, pentene, and/or C₆⁺ olefinic hydrocarbons. In one embodiment, the gaseous feed mixture may comprise one or more C₂⁺ olefinic hydrocarbons, selected from the group consisting of ethylene, propylene, butene, pentene, and combinations of these. Any of these olefinic hydrocarbons, or combination of olefinic hydrocarbons, may be present, for example, in an amount, or total (combined) amount, of at least about 0.3 mol-% (e.g., from about 0.3 mol-% to about 15 mol-%), such as at least about 1 mol-% (e.g., from about 1 mol-% to about 10 mol-%). In general, any one or more hydrocarbons other than CH₄ may be present in the gaseous feed mixture in an amount, or in a total (combined) amount, of at least about 3 mol-% (e.g., from about 3 mol-% to about 45 mol-%), such as at least about 5 mol-% (e.g., from about 5 mol-% to about 30 mol-%). In terms of their sulfur tolerance, reforming/RWGS catalysts described herein provide further advantages associated with the ability to process sulfur-containing gaseous feed mixtures, such as those comprising or being derived from natural gas that, depending on its source, may contain sulfur in the form of H₂S or other sulfur-bearing components. In general, the gaseous feed mixture may comprise at least about 1 mole-ppm (e.g., from about 1 mol-ppm to about 1 mol-%) total sulfur (e.g., present as H₂S and/or other sulfur-bearing components), such as at least about 3 mol-ppm (e.g., from about 3 mol-ppm to about 5000 mol-ppm) of total sulfur, at least about 10 mol-ppm (e.g., from about 10 mol-ppm to about 1000 mol-ppm of total sulfur, or at least about 100 mol-ppm (e.g., from about 100 mol-ppm to about 1000 mol-ppm) of total sulfur.

Improvements in the stability of reforming/RWGS catalysts described herein, particularly with respect to gaseous feed mixtures comprising non-CH₄ hydrocarbons and/or sulfur-bearing components as described herein that generally promote catalyst deactivation, may be attributed at least in part to their high activity, which manifests in lower operating (reactor or catalyst bed) temperatures. This, in turn, contributes to a reduced rate of the formation and deposition of coke on the catalyst surface and an extended, stable operation. In view of the ability of reforming/RWGS catalysts described herein to achieve a given or targeted level of performance (e.g., in terms of CH₄ conversion) at a relatively low operating (or average catalyst bed) temperature as a reforming/RWGS condition, such catalysts may alternatively be referred to as “cool” reforming catalysts, with the associated processes being referred to as “cool” reforming processes.

Representative reforming/RWGS catalysts suitable for catalyzing the reforming and/or RWGS reactions described herein comprise a noble metal, and possibly two, or even more than two, noble metals, on a solid support. The solid support may comprise cerium oxide, or, more particularly, cerium oxide in combination with a suitable binder (e.g., alumina) in a suitable amount (e.g., from about 5 wt-% to about 35 wt-%) to impart mechanical strength.

The phrase “on a solid support” is intended to encompass catalysts in which the active metal(s) is/are on the support surface and/or within a porous internal structure of the support. The solid support preferably comprises a metal oxide, with cerium oxide being of particular interest. Cerium oxide may be present in an amount of at least about 60 wt-% and preferably at least about 75 wt-%, based on the weight of the solid support (e.g., relative to the total amount(s) of metal oxide(s) in the solid support). Whether or not in oxide form, cerium may be present in an amount from about 30 wt-% to about 80 wt-%, and preferably from about 40 wt-% to about 65 wt-%, of the catalyst. The solid support may comprise all or substantially all (e.g., greater than about 95 wt-%) cerium oxide, or otherwise all or substantially all (e.g., greater than about 95 wt-%) of a combined amount of cerium oxide and a second metal oxide (e.g., aluminum oxide) that acts as a binder. According to particular embodiments, the reforming/RWGS catalyst may comprise a noble metal such as Pt on a solid support comprising cerium oxide in an amount as described above (e.g., at least about 60 wt-%), with aluminum oxide representing all or substantially all of balance of the solid support (e.g., cerium oxide and aluminum oxide being present in a combined amount of at least about 95 wt-% of the solid support), with this amount of aluminum oxide possibly corresponding to all or substantially all of the balance of the reforming/RWGS catalyst, excluding metals (e.g., Pt) deposited on the solid support. A representative solid support may comprise at least about 70 wt-%, or at least about 75 wt-%, of cerium oxide, with at least about 10 wt-% or at least about 15 wt-%, being aluminum oxide, with the latter component of the solid support being a relatively non-acidic metal oxide that adds mechanical strength.

In the solid support, one or more of metal oxides other than cerium oxide, such as aluminum oxide, silicon oxide, titanium oxide, zirconium oxide, magnesium oxide, calcium oxide, iron oxide, vanadium oxide, chromium oxide, nickel oxide, tungsten oxide, strontium oxide, etc., may also be present, independently in individual amounts, or otherwise in combined amounts in the case of two or more of such other metal oxides, representing a minor portion, such as less than about 50 wt-%, less than about 30 wt-%, less than about 10 wt-%, or less than about 5 wt-%, of the solid support. Preferably, one or more of silicon oxide, titanium oxide, zirconium oxide, magnesium oxide, calcium oxide, iron oxide, vanadium oxide, chromium oxide, nickel oxide, tungsten oxide, and strontium oxide is substantially absent in the solid support. For example, these metal oxides may be present, independently in individual amounts, or otherwise in combined amounts in the case of two or more of such other metal oxides, of less than about 3 wt-%, less than about 0.5 wt-%, or even less than about 0.1 wt-%, of the solid support. For illustrative purposes, in specific embodiments, (i) silicon oxide (silica) may be present in an amount of less than about 0.5 wt-% of the solid support, (ii) nickel oxide may be present in amount of less than about 0.5 wt-% of the solid support, or (iii) silicon oxide and nickel oxide may be present in a combined amount of less than about 0.5 wt-% of the solid support. In other embodiments, the solid support may comprise one or more of such other metal oxides, including aluminum oxide, independently in individual amounts, or otherwise in combined amounts in the case of two or more of such other metal oxides, representing a major portion, such as greater than about 50 wt-%, greater than about 70 wt-%, or greater than about 90 wt-%, of the solid support. In such cases, the solid support may also optionally comprise cerium oxide in an amount representing a minor portion, such as less than about 50 wt-%, less than about 30 wt-%, or less than about 10 wt-%, of the solid support. Such minor portion of cerium oxide may also represent all or substantially all of the balance of the solid support, which is not represented by the one or more of such other metal oxides.

According to particular embodiments, the solid support may comprise, in addition to cerium oxide, a second metal oxide that acts as a binder for cerium oxide. Such second metal oxide may be selected from the group of other metal oxides described above, namely, aluminum oxide, silicon oxide, titanium oxide, zirconium oxide, magnesium oxide, calcium oxide, iron oxide, vanadium oxide, chromium oxide, nickel oxide, tungsten oxide, and strontium oxide. Such second metal oxide may be present in an amount generally from about 1 wt-% to about 45 wt-%, typically from about 5 wt-% to about 35 wt-%, and often from about 10 wt-% to about 25 wt-%, of the solid support. Preferably, the solid support comprises cerium oxide and the second metal oxide in a combined amount of generally at least about 85 wt-%, typically at least about 95 wt-%, and often at least about 99 wt-%, of the solid support. The solid support may comprise cerium oxide and the second metal oxide in a combined amount of generally at least about 85 wt-%, typically at least about 92 wt-%, and often at least about 95 wt-%, of the reforming/RWGS catalyst. A preferred second metal oxide that acts as a binder for cerium oxide is aluminum oxide.

A preferred property of the solid support (e.g., comprising predominantly cerium oxide), and consequently the reforming/RWGS catalyst, is low acidity. In this regard, excessive acid sites on the support or catalyst, and in particular strong, Bronsted acid sites, are believed to contribute to coking and catalyst deactivation during the reforming and/or RWGS reactions. Importantly, advantages of a low Bronsted acid site proportion, or concentration, in terms of establishing a commercially feasible catalyst life, are gained despite the fact that strong acid sites are known to promote the activity of a number of significant commercial reactions. An extensively used method for acid site strength determination and quantification with respect to solid materials is temperature programmed desorption using ammonia as a molecular probe (NH₃-TPD). According to this method, a sample of the solid material is prepared by degassing and activation at elevated temperature and in an inert environment, in order to remove water and other bound species. The sample is then saturated with NH₃, with the saturation temperature (e.g., 100° C.) and subsequent purge with an inert gas (e.g., helium) providing conditions that remove any physisorbed NH₃. Temperature programmed desorption of the activated and saturated sample is initiated by ramping the temperature at a predetermined rate (e.g., 10° C/minute) to a final temperature (e.g., 400° C.) under the flow of the inert gas. The concentration of NH₃ in this gas is continually measured as it is driven from acid sites of the solid material having increasing strengths that correspond to increasing desorption temperatures. The determination of NH₃ concentration in the flowing inert gas can be performed, for example, using gas chromatography with a thermal conductivity detector (GC-TCD).

Typically, the NH₃ concentration versus temperature profile will include peaks at low and high temperatures that correspond to sites of the solid material having comparatively low and high acid strengths, respectively. The areas under these peaks can then provide relative concentrations of acid sites of the differing types of acid strength (e.g., expressed as a percentage of total acid sites), or otherwise these areas can be used to determine the absolute concentrations of the differing types (e.g., expressed in terms of milliequivalents per gram of the solid material). In the case of a solid support or reforming/RWGS catalyst that generates two peaks on the NH₃ concentration versus temperature profile over a relevant range, for example from 100° C. to 400° C., a first, low temperature peak may be associated with weak Lewis acid sites, whereas a second, high temperature peak may be associated with strong, Bronsted acid sites. For representative solid supports (e.g., comprising predominantly cerium oxide) as well as reforming/RWGS catalysts having such supports (in view of the relatively small or negligible impact, on the NH₃-TPD analysis, of catalytically active metals being deposited on such supports), the NH₃ concentration versus temperature profile obtained from an NH₃-TPD analysis over a temperature range from 100° C. to 400° C. (with such profile having, for example, two identifiable peaks) may exhibit a maximum NH₃ concentration at a temperature of less than about 300° C. (e.g., from about 150° C. to about 300° C.), and more typically at a temperature of less than about 250° C. (e.g., from about 150° C. to about 250° C.). This maximum NH₃ concentration may therefore be associated with a low temperature peak corresponding to weak Lewis acid sites, with the maximum NH₃ concentration and temperature at which this concentration is exhibited defining a point on this low temperature peak. Based on a peak area of this low temperature peak, relative to a peak area of a higher temperature peak corresponding to strong, Bronsted acid sites, the Lewis acid sites may represent at least about 25%, at least about 30%, or at least about 35%, of the total acid sites (e.g., the total Lewis and Bronsted acid sites combined). The higher temperature peak may, for example, exhibit a maximum NH₃ concentration at a temperature from about 300° C. to about 350° C., or, more typically, from about 300° C. to about 325° C. The maximum NH₃ concentration associated with the low temperature peak is normally greater than the maximum NH₃ concentration associated with the higher temperature peak, as a further indication that weak Lewis acid sites contribute to a substantial proportion of the overall acid sites of the solid support or reforming/RWGS catalyst. In representative embodiments, the solid support or reforming/RWGS catalyst may have a Lewis acid site concentration of at least about 0.25 milliequivalents per gram (meq/g) (e.g., from about 0.25 meq/g to about 1.5 meq/g), and more typically at least about 0.35 milliequivalents per gram (meq/g) (e.g., from about 0.35 meq/g to about 0.85 meq/g).

The solid support (e.g., comprising predominantly cerium oxide), as well as the reforming/RWGS catalyst comprising such support, may have a surface area from about 1 m²/g to about 100 m²/g, such as from about 10 m²/g to about 50 m²/g. Surface area may be determined according to the BET (Brunauer, Emmett and Teller) method based on nitrogen adsorption (ASTM D1993-03(2008)). The support and/or catalyst may have a total pore volume, of pores in a size range of 1.7-300 nanometers (nm), from about 0.01 cc/g to about 0.5 cc/g, such as from about 0.08 cc/g to about 0.25 cc/g. Pore volume may be measured by mercury porosimetry. The support and/or catalyst may have an average pore diameter from about 2 to about 75 nm, such as from about 5 to about 50 nm. The support and/or catalyst may have (i) from about 10% to about 80%, such as from about 30% to about 55%, of its pore volume attributed to macropores of >50 nm, (ii) from about 20% to about 85%, such as from about 35% to about 60%, of its pore volume attributed to mesopores of 2-50 nm, and/or (iii) less than about 2%, such as less than about 0.5%, of its pore volume attributed to micropores of <2 nm. Pore size distribution may be obtained using the Barrett, Joyner, and Halenda method.

Noble metals are understood as referring to a class of metallic elements that are resistant to oxidation. In representative embodiments, the noble metal, and in some cases at least two noble metals, of the reforming/RWGS catalyst may be selected from the group consisting of platinum (Pt), rhodium (Rh), ruthenium (Ru), palladium (Pd), silver (Ag), osmium (Os), iridium (Ir), and gold (Au), with the term “consisting of” being used merely to denote group members, according to a specific embodiment, from which the noble metal(s) are selected, but not to preclude the addition of other noble metals and/or other metals generally. Accordingly, a catalyst comprising a noble metal embraces a catalyst comprising at least two noble metals, as well as a catalyst comprising at least three noble metals, and likewise a catalyst comprising two noble metals and a third, non-noble metal such as a promoter metal (e.g., a transition metal). According to preferred embodiments, the noble metal is present in an amount, or alternatively the at least two noble metals are each independently present in amounts, from about 0.05 wt-% to about 5 wt-%, from about 0.1 wt-% to about 3 wt-%, from about 0.3 wt-% to about 1 wt-%, or from about 0.5 wt-% to about 2 wt-%, based on the weight of the catalyst. For example, a representative reforming/RWGS catalyst may comprise the noble metal Pt, the noble metal Rh, or the two noble metals Pt and Rh in combination, with such noble metal(s) being present independently in an amount within any of these ranges (e.g., from about 0.05 wt-% to about 5 wt-%), or otherwise in a combined amount within any of these ranges. That is, either the Pt may be present in such an amount, the Rh may be present in such an amount, or both Pt and Rh in combination may be present in such an amount. A preferred, noble metal-containing reforming/RWGS catalyst comprises one or both of Pt and Rh, either of which, whether used alone or in combination, may be present in an amount from about 0.3 wt-% to about 1 wt-%, on a support comprising, comprising substantially all, or consisting essentially of, cerium oxide and optionally a metal oxide binder (e.g., aluminum oxide) as described above. As a noble metal, Pt is particularly preferred. Regardless of the noble metal(s) used or the particular amounts used, preferably these noble metals are in their elemental (metallic or zero oxidation state) form. For example, with respect to the preferred, noble metal-containing reforming/RWGS catalyst described above, such catalyst may comprise one or both of Pt and Rh, either of which, whether used alone or in combination, may be present in its respective elemental form in an amount from about 0.3 wt-% to about 1 wt-%, based on the weight of the catalyst. Whereas other (compound) forms of Pt and/or Rh may also be present, preferably Pt and/or Rh in non-elemental forms, or noble metals generally in non-elemental forms, are present independently in individual amounts, or otherwise in combined amounts in the case of two or more noble metals, of less than about 1 wt-%, less than about 0.5 wt-%, or even less than about 0.1 wt-%, of the reforming/RWGS catalyst.

In representative embodiments, one or two noble metals (e.g., Pt and/or Rh) may be substantially the only one or two noble metals present in the reforming/RWGS catalyst, such that, for example, any other noble metal(s) is/are present in an amount or a combined amount of less than about 0.1 wt-%, or less than about 0.05 wt-%, based on the weight of the catalyst. In further representative embodiments, one or two noble metals (e.g., Pt and/or Rh) are substantially the only metals present in the catalyst, with the exception of metals present in the solid support (e.g., such as cerium being present in the solid support as cerium oxide). For example, any other metal(s), besides one or two noble metals and metals of the solid support, may be present in an amount or a combined amount of less than about 0.1 wt-%, or less than about 0.05 wt-%, based on the weight of the catalyst. In some embodiments, certain metals may be substantially absent in the catalyst, whether in elemental form or in compound form (e.g., in the form of an oxide as a metal oxide component of the solid support). For example, certain metals may impart unwanted acidity in the solid support, provide insubstantial catalytic activity, and/or catalyze undesired reactions. In particular embodiments, one or more of Si, Ti, Zr, Mg, Ca, Fe, V, Cr, Ni, W, and Sr is substantially absent in the solid support. For example, these metals may be present, independently in individual amounts, or otherwise in combined amounts in the case of two or more of such metals, of less than about 0.5 wt-%, less than about 0.1 wt-%, or even less than about 0.05 wt-%, of the reforming/RWGS catalyst, or of the solid support for the catalyst. For example, one or more of Si, Zr, Mg, and Ni may be present in these individual amounts or combined amounts. Any metals present in the catalyst, including noble metal(s), may have a metal particle size in the range generally from about 0.3 nanometers (nm) to about 20 nm, typically from about 0.5 nm to about 10 nm, and often from about 1 nm to about 5 nm.

The noble metal(s) may be incorporated in the solid support according to known techniques for catalyst preparation, including sublimation, impregnation, or dry mixing. In the case of impregnation, which is a preferred technique, an impregnation solution of a soluble compound of one or more of the noble metals in a polar (aqueous) or non-polar (e.g., organic) solvent may be contacted with the solid support, preferably under an inert atmosphere. For example, this contacting may be carried out, preferably with stirring, in a surrounding atmosphere of nitrogen, argon, and/or helium, or otherwise in a non-inert atmosphere, such as air. The solvent may then be evaporated from the solid support, for example using heating, flowing gas, and/or vacuum conditions, leaving the dried, noble metal-impregnated support. The noble metal(s) may be impregnated in the solid support, such as in the case of a single noble metal (e.g., Pt) being impregnated, or in the case of two noble metals being impregnated simultaneously with both being dissolved in the same impregnation solution, or otherwise being impregnated (e.g., sequentially) using different impregnation solutions and contacting steps. In any event, the noble metal-impregnated support may be subjected to further preparation steps, such as washing with the solvent to remove excess noble metal(s) and impurities, further drying, calcination, etc. to provide the reforming/RWGS catalyst.

The solid support itself may be prepared according to known methods, such as extrusion to form cylindrical particles (extrudates) or oil dropping or spray drying to form spherical particles. Regardless of the specific shape of the solid support and resulting catalyst particles, the amounts of noble metal(s) being present in the catalyst, as described above, refer to the weight of such noble metal(s), on average, in a given catalyst particle (e.g., of any shape such as cylindrical or spherical), independent of the particular distribution of the noble metal(s) within the particle. In this regard, it can be appreciated that different preparation methods can provide different distributions, such as deposition of the noble metal(s) primarily on or near the surface of the solid support or uniform distribution of the noble metal(s) throughout the solid support. In general, weight percentages described herein, being based on the weight of the solid support or otherwise based on the weight of catalyst, can refer to weight percentages in a single catalyst particle but more typically refer to average weight percentages over a large number of catalyst particles, such as the number in a catalyst bed within a reactor that is used in a first or initial stage for carrying out reforming and/or RWGS.

### Reforming/RWGS Conditions

In the first or initial stage, reforming and/or RWGS reactions, and preferably both simultaneously, are performed by contacting a gaseous feed mixture, preferably continuously using a flowing stream of the gaseous feed mixture to improve process efficiency, with reforming/RWGS catalyst as described herein. For example, contacting may be performed by continuously flowing the gaseous feed mixture through a reactor (which may be referred to as a reforming/RWGS reactor) that contains a noble metal-containing reforming/RWGS catalyst as described herein. The reactor is maintained under reforming/RWGS conditions, which are namely the conditions within a reactor vessel and, more particularly, within a bed of the reforming/RWGS catalyst that is contained in the vessel. These conditions include a temperature, pressure, and flow rate for the effective conversion of methane, and optionally other hydrocarbons, to hydrogen, in case such conditions are used to carry out reforming. Alternatively, but preferably in combination, these conditions are effective for the conversion of CO₂ to CO and thereby carry out the RWGS reaction.

Reforming/RWGS conditions that are useful for one or both of these reactions include a temperature generally from about 649° C. (1200° F.) to about 927° C. (1700° F.), typically from about 725° C. (1337° F.) to about 900° C. (1652° F.), and often from about 750° C. (1382° F.) to about 880° C. (1616° F.). In preferred embodiments, processes described herein, by virtue of the high activity of the catalyst, can effectively reform (oxidize) a hydrogen source as described herein (e.g., CH₄ and/or possibly other hydrocarbons such as C₂H₆ and/or C₃H₈) and/or perform the RWGS reaction at significantly lower temperatures, compared to a representative conventional reforming temperature of 816° C. (1500° F.). For example, the reforming/RWGS conditions can include a temperature in a range from about 677° C. (1250° F.) to about 788° C. (1450° F.), or from about 704° C. (1300° F.) to about 760° C. (1400° F.). In the case of dry reforming that occurs if the gaseous teed mixture contains CO₂ as an oxidant for reforming, with relatively little or no H₂O and/or 02, higher temperatures may be used, for example from about 843° C. (1550° F.) to about 1010° C. (1850° F.), or from about 885° C. (1625° F.) to about 941° C. (1725° F.). The presence of H₂S and/or other sulfur-bearing contaminants in significant concentrations (e.g., 100-1000 mol-ppm) may warrant increased temperatures, for example in a range from about 732° C. (1350° F.) to about 843° C. (1550° F.), or from about 760° C. (1400° F.) to about 816° C. (1500° F.), to maintain desired conversion levels (e.g., a CH₄ conversion of greater than about 85%). Advantageously, it has been discovered that the compensating effect of increasing temperature in response to increased sulfur concentrations in the gaseous feed mixture does not adversely affect catalyst stability. That is, the overall catalyst life is essentially unchanged, with respect to a comparison between a baseline sulfur-free operation and a sulfur-containing operation performed at a higher, compensating temperature.

Particularly in the case of large-scale operation, reactors operate with a limited release of heat to their surroundings (e.g., in the case of adiabatic operation), such that the catalyst bed temperature may vary as a given reaction proceeds (e.g., a fixed bed temperature profile may be characterized by an increasing or decreasing profile along the axial length of the reactor in the case of an exothermic or endothermic reaction, respectively). Accordingly, temperatures given herein that are associated with reforming/RWGS conditions, or otherwise downstream FT reaction conditions and/or cracking reaction conditions, should be understood to mean average (or weighted average) catalyst bed temperatures. However, in view of the high activity of catalyst compositions described herein, particularly with respect to reforming/RWGS catalysts, temperatures given herein, and particularly those that are associated with reforming/RWGS conditions, in some embodiments may be maximum or peak catalyst bed temperatures.

Yet other reforming/RWGS conditions can include an above-ambient pressure, i.e., a pressure above a gauge pressure of 0 kPa (0 psig), corresponding to an absolute pressure of 101 kPa (14.7 psia). Because the reforming reactions make a greater number of moles of product versus moles of reactant, in some cases equilibrium may be favored at relatively low pressures. Representative reforming/RWGS conditions can include a gauge pressure generally from about 0 kPa (0 psig) to about 2.00 MPa (290 psig), typically from about 100 kPa (15 psig) to about 1.50 MPa (218 psig), and often from about 500 kPa (73 psig) to about 1.00 MPa (145 psig). According to some embodiments, it may be desirable to operate at higher pressures, for example in the range from about 207 kPa (30 psig) to about 5.2 MPa (750 psig), such as from about 1.4 MPa (200 psig) to about 3.4 MPa (500 psig). For example, a gaseous effluent recycled from a downstream FT reaction or optional wax cracking or isomerization may have a pressure P_{recycle} that is greater than 2.1 MPa (300 psig), and to minimize the energy loss associated with reducing the pressure of such recycle stream, in such case the reforming/RWGS pressure may be increased so that it is close to or the same as P_{recycle} (e.g., the reforming/RWGS reactor pressure may be at least 50% of P_{recycle}, or at least 75% of P_{recycle}, 90% of P_{recycle}, or at least 95% of P_{recycle}). Representative reforming/RWGS conditions may further include a WHSV generally from about 0.05 hr⁻¹ to about 10 hr⁻¹, typically from about 0.1 hr⁻¹ to about 8.0 hr⁻¹, and often from about 0.5 hr⁻¹ to about 5.0 hr⁻¹. As is understood in the art, the WHSV is the weight flow of the gaseous feed mixture (or total weight flow of all inputs, or gaseous feed mixture components as described above, to one or more reactors used in the reforming stage or RWGS stage) divided by the total weight of catalyst in the reforming/RWGS reactor(s) and represents the equivalent catalyst bed weights of the gaseous feed mixture (or all inputs or components) processed per hour. The WHSV is related to the inverse of the reactor residence time. The reforming/RWGS catalyst may be contained within the reactor(s) in the form of a fixed bed, but other catalyst systems are also possible, such as moving bed and fluidized bed systems that may be beneficial in processes using continuous catalyst regeneration. Regardless of the particular bed configuration, preferably the catalyst bed comprises discreet particles of refomling/RWGS catalyst, as opposed to a monolithic form of catalyst. For example, such discreet catalyst particles may have a spherical or cylindrical diameter of less than about 10 mm and often less than about 5 mm (e.g., about 2 mm or about 3 mm). in the case of cylindrical catalyst particles (e.g., formed by extrusion), these may have a comparable length dimension (e.g., from about 1 mm to about 10 mm, such as about 5 trim).

Advantageously, within any of the above temperature ranges and with respect to gaseous feed mixtures comprising CH₄, the high activity of the catalyst can achieve a conversion of this component of at least about 60% (e.g., from about 60% to about 99%), at least about 75% (e.g., from about 80% to about 99%), at least about 85% (e.g., from about 85% to about 99%), or at least about 90% (e.g., from about 90% to about 97%). A desired conversion level, with respect to a given gaseous feed mixture and reforming/RWGS catalyst, may be attained or controlled by adjusting the particular reactor or catalyst bed temperature and/or other reforming/RWGS conditions (e.g., WHSV and/or pressure) as would be appreciated by those having skill in the art, with knowledge gained from the present disclosure. Advantageously, noble metal-containing catalysts as described herein may be sufficiently active to achieve a significant CH₄ conversion, such as at least about 60% or at least about 75%, in a stable manner at a temperature of at most about 732° C. (1350° F.), or even at most about 704° C. (1300° F.) (e.g., as a peak or maximum catalyst bed temperature). In the case of dry reforming, for example if the oxidant for reforming (according to the composition of the gaseous feed mixture) is predominantly, substantially all, or all CO₂ as described above, such CH₄ conversion levels may be achieved at higher temperatures, for example at most about 918° C. (1685° F.), or in some cases at most about to about 885° C. (1625° F.) (e.g., as a peak or maximum catalyst bed temperature). As is understood in the art, the conversion of CH₄ can be calculated on the basis of:

100*(CH4_{feed}−CH4_{prod})/CH4_{feed},

wherein CH4_{feed} is the total amount (e.g., total weight or total moles) of CH₄ in the gaseous feed mixture (or total amount in all inputs or in all gaseous feed mixture components) provided to one or more reactors used in the reforming stage or RWGS stage and CH4_{prod} is the total amount of CH₄ in the synthesis gas intermediate obtained from this stage. In the case of continuous processes, these total amounts may be more conveniently expressed in terms of flow rates, or total amounts per unit time (e.g., total weight/hr or total moles/hr). The same or higher levels of conversion may be achieved with respect to other hydrocarbons, such as C₂H₆ and/or C₃H₈, which may be present in the gaseous feed mixture, in place of CH₄ but more preferably in combination with CH₄.These C₂ and/or C₃ hydrocarbons are generally more easily converted to the synthesis gas intermediate, relative to CH₄, under a given set of reforming/RWGS conditions. The conversions of C₂H₆ and/or C₃H₈ can be determined in a manner analogous to that described above with respect to the determination of CH₄ conversion. These conversion levels of CH₄, C₂H₆, and/or C₃H₈,may be based on “per-pass” conversion, achieved in a single pass through a reforming/RWGS stage (e.g., a reforming/RWGS reactor of this stage), or otherwise based on overall conversion, achieved by returning a recycle portion of the FT synthesis effluent, or of a polishing effluent, back to the reforming/RWGS stage (e.g., a reforming/RWGS reactor of this stage), as described herein. In this regard, a recycle portion of (i) a fraction enriched in H₂ and CO₂ or (ii) a fraction enriched in the hydrogen source and CO₂ (or even a part of such fraction (i) or (ii)) may contain unconverted CH₄, C₂H₆ and/or C₃H₈ that can be converted in successive passes through the first reaction stage, thereby increasing the conversion of these light hydrocarbons on an overall basis. Similarly, overall conversion of hydrocarbons may be increased relative to the per-pass conversion, by returning a recycle portion of the FT synthesis effluent, or of a polishing effluent, that is namely a hydrocarbon recycle obtained from all or a portion of a separated fraction, such as a separated fraction enriched in naphtha-boiling range hydrocarbons, which may alternatively be referred to as a naphtha-boiling range hydrocarbon fraction. To the extent such fraction may contain amounts of CH₄, C₂H₆ and/or C₃H₈,the use of a hydrocarbon recycle may likewise increase the conversion of these light hydrocarbons on an overall basis.

In view of reforming reactions (e.g., the reforming of CH₄, C₂H₆, and/or C₃H₈) producing both H₂ and CO, the concentration of both of these components may be increased in the synthesis gas intermediate (product of reforming), relative to the gaseous feed mixture (or combined inputs, or gaseous feed mixture components, fed to one or more reactors used in the reforming stage or RWGS stage). An increase in CO concentration may also result from the RWGS reaction, either alone or in combination with reforming, in the case of CO₂ being present in the gaseous mixture. In this regard, the extent of the RWGS reaction, according to which CO₂ is converted to CO with equilibrium constraints, may be characterized by a conversion of CO₂ in a reforming stage or an RWGS stage of at least about 60%, at least about 70%, or at least about 80%, determined in a manner analogous to that described above with respect to the determination of CH₄ conversion. In some embodiments, depending on the H₂ concentration in the gaseous feed mixture and the extent of the RWGS reaction, the concentration of CO may be increased, whereas the concentration of H₂ may be decreased. In representative embodiments, the synthesis gas intermediate may comprise CO in an amount of at least about 5 moi-% (e.g., from about 5 mot-% to about 50 mol-%) or at least about 8 mol -% (e.g., from about 8 mot-% to about 35 mol-%). In other embodiments, according to which high levels of conversion of CH₄, C₂H₆, and/or C₃H₈ are achieved, the synthesis gas intermediate may comprise CO in a higher amount, such as at least about 30 mol-% (e.g., from about 30 moi-P/0 to about 65 mol-%) or at least about 40 moi-% (e.g., from about 40 mol-% to about 55 moi-%). In further representative embodiments, the synthesis gas intermediate may comprise E12 in an amount of at least about 30 mol-% (e.g., from about 30 mot-% to about 90 mol-%) or at least about 40 mol-% (e.g., from about 40 mol-% to about 80 mol-%). With respect to the gaseous feed mixture, depending on the amount of H₂ present, as well as amounts of the oxidants CO₂ and H₂O present (which react ; for example, with CH₄ to yield 1:1 and 3:1 stoichiornetric molar ratios of H₂:CO, respectively) the H₂:CO molar ratio of the synthesis gas intermediate may be from about 1.0 to about 7.0, such as from about 4.0 to about 6.5, in the case of high ratios. Otherwise, in the case of lower ratios, the H₂:CO molar ratio of the synthesis gas intermediate may be from about 1.0 to about 3.0, such as from about 1.8 to about 2.4 or from about 2.1 to about 2.7. According to yet other embodiments, for example in the case of reforming CH₄, C₂H₆, and/or C₃H₈ with an oxidant that may be predominantly, substantially all, or all CO₂, the H₂:CO molar ratio of the synthesis gas intermediate may be less, in view of the stoichiometry of the dry reforming reaction alone. For example, this 112:CO molar ratio may be from about 0.5 to about 1.5, such as from about 0.8 to about 1.2. According to still further embodiments, the H₂:CO molar ratio of the synthesis gas intermediate may be “tuned” using the amount of H₂O (steam) being input to the gaseous mixture as a “handle.” For example, more or less H₂O may be added to obtain a desired or setpoint H₂:CO molar ratio, with such setpoi.nt being a discreet value within any of the ranges above. Operation of the reforming stage or RWGS stage may include, for example, tuning the H₂:CO molar ratio of the synthesis gas intermediate to a value from about 2.1 to about 2.5, with higher amounts of steam input corresponding to higher molar ratios. According to still further embodiments, in which the gaseous mixture comprises a fresh makeup feed that further comprises H₂, adjusting the amount of such H₂ in such fresh makeup feed may be yet another “handle” to “tune” the H₂:CO molar ratio of the synthesis gas intermediate instead of or in combination with adjusting the amount of steam being input the gaseous mixture.

In any event, molar ratios as described above may be representative of the synthesis gas intermediate or portion thereof used for FT synthesis, as obtained directly from a reactor used in the reforming stage or the RWGS stage, or otherwise as obtained following an adjustment of the H₂:CO molar ratio, according to an intervening operation, for example by adding a source of Hz and/or a source of CO to this intermediate or portion thereof, prior to (e.g., upstream of) the FT synthesis stage. A representative source of Hz and/or CO is a recycle portion of (i) a fraction of the FT synthesis effluent that enriched in Hz and CO₂ or (ii) a fraction of the FT synthesis effluent that is enriched in the hydrogen source and CO₂ (or even a part of such fraction (i) or (ii)), as described herein. Another representative source of H₂ is hydrogen that has been purified (e.g., by PSA or membrane separation), and another representative source of H₂ and CO is unpurified hydrogen resulting from steam methane reforming (e.g., syngas). In other embodiments, between (a) the reforming stage or RWGS stage and (b) the FT synthesis stage, water may be removed (e.g., condensed) from the synthesis gas intermediate or portion thereof used for FT synthesis.

### Fischer-Tropsch (FT) Synthesis

The first or initial reaction stage, as described above, according to which a synthesis gas intermediate comprising both H₂ and CO (i.e., an H₂/CO mixture) is produced, may be followed by a second stage of converting this synthesis gas intermediate to C₄⁺ hydrocarbons that are contained in the liquid hydrocarbon product. The second stage generally involves Fischer-Tropsch (FT) synthesis to produce hydrocarbons of a higher molecular weight, according to the reaction:

(2n+1) H₂+n CO→CₙH₂ₙ₊₂+n H₂O.

Specifically, the FT synthesis reaction may be used to produce alkane hydrocarbons having two or more carbon atoms, with a distribution of their specific numbers of carbon numbers as described above. Representative processes may comprise converting all or a portion of the synthesis gas intermediate via FT synthesis, optionally following one or more intervening operations performed on this intermediate that may be used to provide an FT feed having a composition and/or properties differing from that/those of the synthesis gas intermediate. Such intervening operations include cooling, heating, pressurizing, depressurizing, separation of one or more components (e.g., removal of condensed water), addition of one or more components (e.g., addition of H₂ and/or CO to adjust the H₂:CO molar ratio of a FT feed relative to that of the synthesis gas intermediate), and/or reaction of one or more components (e.g., reaction of H₂ and/or CO using a separate water-gas shift reaction or reverse water-gas shift reaction), which operation(s) is/are performed on the synthesis gas intermediate to provide an FT feed to FT reactor(s) of an FT synthesis stage. In view of the temperatures and pressures typically used in the FT reactor(s) of the FT synthesis stage relative to those used in the reactor(s) of the reforming stage or RWGS stage, the synthesis gas intermediate may be cooled, separated from condensed water, and pressurized. In some embodiments, these may be the only intervening operations to which the synthesis gas intermediate is subjected, to provide the FT feed. In other embodiments, cooling and pressurizing may be the only intervening operations. In yet other embodiments, intervening operations that may be omitted include drying of the synthesis gas intermediate to remove vapor phase H20 (which is therefore different from condensing liquid phase H₂O and can include, e.g., using a sorbent selective for water vapor, such as 5A molecular sieve) and/or CO₂ removal according to conventional acid gas treating steps (e.g., amine scrubbing). In yet other embodiments, an intervening operation may be the addition of (i.e., the combination of the synthesis gas intermediate or portion thereof with) (i) a fraction of the FT synthesis effluent that is enriched in H₂ and CO₂ or (ii) a fraction of the FT synthesis effluent that is enriched in the hydrogen source and CO₂ (or portions of (i) or (ii), such as a recycle portion thereof). According to some embodiments, CO₂ removal may be performed on the synthesis gas intermediate, upstream of the FT synthesis stage (e.g., as an intervening operation). Preferably, prior to the FT reactor(s), water produced in the reactor(s) of the reforming stage or RWGS stage is condensed from the synthesis gas intermediate, and/or also preferably the H₂:CO molar ratio of the synthesis gas intermediate is not adjusted. The use of no intervening operations between the reforming stage or RWGS stage and the FT synthesis stage, limited intervening operations, and/or the omission or certain intervening operations, results in advantages associated with the overall simplification of processes for producing liquid hydrocarbon products.

Conditions in the FT synthesis stage, and more particularly FT reactor(s) used in this stage, are suitable for the conversion of H₂ and CO to C₄⁺ hydrocarbons. In representative embodiments, FT reaction conditions, suitable for use in at least one FT reactor or, more particularly, a catalyst bed contained in such reactor, can include an FT reaction temperature in a range from about 121° C. (250° F.) to about 288° C. (550° F.), or from about 193° C. (380° F.) to about 260° C. (500° F.). An FT reaction pressure can include a gauge pressure from about 621 kPa (90 psig) to about 5.00 MPa (725 psig), or from about 2.50 MPa (362 psig) to about 3.50 MPa (508 psig).

In the FT reactor(s), an FT feed, representing all or a portion of the synthesis gas intermediate, optionally following one or more intervening operations described above, may be contacted with a suitable FT catalyst (e.g., bed of FT catalyst particles disposed within the FT reactor) under FT reaction conditions, which may include the temperatures and/or pressures as described above. Representative FT catalysts comprise, as FT active metal(s), one or more transition metals selected from cobalt (Co), iron (Fe), ruthenium (Ru), and nickel (Ni). A preferred FT catalyst comprises generally at least about 5 wt-% of the transition metal(s), typically about 5 wt-% to about 15 wt-% of the transition metal(s), and often at least about 15 wt-% of the transition metal(s), on a solid support. The phrase “on a solid support” is intended to encompass catalysts in which the active metal(s) is/are on the support surface and/or within a porous internal structure of the support. Representative solid supports comprise one or more metal oxides, selected from the group consisting of aluminum oxide, silicon oxide, titanium oxide, zirconium oxide, magnesium oxide, strontium oxide, etc. The solid support may comprise all or substantially all (e.g., greater than about 95 wt-%) of the one or more of such metal oxides. Preferred FT catalysts comprise the transition metal cobalt (Co) in the above amounts (e.g., at least about 10 wt-%) on a support comprising aluminum oxide (alumina).

The FT catalysts and FT reaction conditions described herein are generally suitable for achieving a conversion of H₂ and/or CO (H₂ conversion or CO conversion) of at least about 20% (e.g., from about 20% to about 99% or from about 20% to about 75%), at least about 30% (e.g., from about 30% to about 95% or from about 30% to about 65%), or at least about 50% (e.g., from about 50% to about 90% or from about 50% to about 85%). These FT conversion levels may be based on H₂ conversion or CO conversion, depending on which reactant is stoichiometrically limited in the FT feed, or in the synthesis gas intermediate, considering the FT synthesis reaction chemistry, and these FT conversion levels may be determined in a manner analogous to that described above with respect to the determination of CH₄ conversion. Preferably, these FT conversion levels are based on CO conversion. These FT conversion levels may be based on “per-pass” conversion, achieved in a single pass through the FT synthesis stage (e.g., an FT reactor of this stage), or otherwise based on overall conversion, achieved by returning a recycle portion of the FT product back to the FT synthesis stage (e.g., an FT reactor of this stage), as described herein.

A desired H₂ conversion and/or CO conversion in the FT reactor(s) may be achieved by adjusting the FT reaction conditions described above (e.g., FT reaction temperature and/or FT reaction pressure), and/or adjusting the weight hourly space velocity (WHSV), as defined above. The FT reaction conditions may include a weight hourly space velocity (WHSV) generally from about 0.01 hr⁻¹ to about 10 hr⁻¹, typically from about 0.05 hr⁻¹ to about 5 hr⁻¹, and often from about 0.3 hr⁻¹ to about 2.5 hr⁻¹. The conversion level (e.g., CO conversion) may be increased, for example, by increasing pressure and decreasing WHSV, having the effects, respectively, of increasing reactant concentrations and reactor residence times. The FT reaction conditions may optionally include returning a recycle portion of the FT product, exiting the FT reactor, back to the FT feed (or possibly back to the synthesis gas intermediate) for combining with the FT feed (or possibly combining with the synthesis gas intermediate), or otherwise back to the FT reactor itself. Recycle operation allows for operation at relatively low “per-pass” conversion through the FT reactor, while achieving a high overall conversion due to the recycle. In some embodiments, this low per-pass conversion may advantageously limit the quantity of high molecular weight hydrocarbons (e.g., normal C₂₀⁺ hydrocarbons) that can be produced as part of the hydrocarbon product distribution obtained from the FT synthesis reaction.

Preferably, however, the FT reaction conditions include relatively little or even no FT product recycle. For example, the FT reaction conditions may include a weight ratio of recycled FT product to FT feed (i.e., a “recycle ratio”), with this recycled FT product and FT feed (e.g., all or a portion of the synthesis gas intermediate) together providing a combined feed to the FT reactor, of generally less than about 1:1, typically less than about 0.5:1, and often less than about 0.1:1. In some cases, the recycle ratio may be 0, meaning that no FT product recycle is used, such that the per-pass conversion is equal to the overall conversion. With such low recycle ratios, a relatively high per-pass H₂ conversion or CO conversion, such as at least about 50% (e.g., from about 50% to about 95%), at least about 60% (e.g., from about 60% to about 92%), or at least about 70% (e.g., from about 70% to about 90%), is desirable in view of process efficiency and economics. As the per-pass conversion level is increased, the distribution of hydrocarbons in the FT product is often shifted to those having increased numbers of carbon atoms.

Embodiments of the invention are therefore directed to a process for producing a liquid hydrocarbon product from a synthesis gas comprising H₂ and CO, for example a synthesis gas intermediate, or an FT feed obtained following one or more intervening operations performed on this intermediate, as described above. The synthesis gas intermediate or FT feed may generally be produced by reforming and/or RWGS reactions, as described above. The process comprises contacting the synthesis gas with an FT catalyst as described herein, such as a catalyst comprising at least about 5 wt-% Co, such as from about 5 wt-% to about 15 wt-% Co or at least about 10 wt-% Co, and/or optionally other transition metal(s), on a solid support, for example a refractory metal oxide such as alumina. The process comprises converting H₂ and CO in the synthesis gas to hydrocarbons, including C₄⁺ hydrocarbons.

Advantageously, in the absence of FT product recycle, compression costs are saved and the overall design of an integrated process may be simplified. To the extent that this requires an increase in the per-pass conversion and associated shift in the distribution of hydrocarbons in the FT product toward those having increased numbers of carbon atoms, including normal C₂₀⁺ hydrocarbons that are solid at room temperature and that are obtained as an undesirable wax fraction, it should be appreciated that aspects of the invention are associated with the discovery of important, further downstream processing strategies for converting these hydrocarbons by cracking to C₄-C₁₉ hydrocarbons, thereby increasing the yield of the liquid hydrocarbon product. In this regard, according to representative processes, the second stage of converting the synthesis gas intermediate to C₄⁺ hydrocarbons comprises a combination of FT synthesis with cracking to reduce the molecular weight of hydrocarbons, particularly by converting normal C₂₀⁺ hydrocarbons obtained from FT synthesis. Rather than cracking alone, a combination of wax cracking and isomerization may be particularly advantageous with respect to: (a) eliminating substantially all normal C₂₀⁺ hydrocarbons, considering that some branched C₂₀⁺ hydrocarbons are more desirable relative to their straight-chain (normal) counterpart, in terms of not contributing to undesirable wax; and (b) improving the properties of the liquid hydrocarbon product, for example, by lowering the freeze point of the jet and diesel fractions by yielding C₄-C₁₉ hydrocarbons with increased branching in their molecular structure.

### FT Synthesis, with Optional Downstream or In-Situ Cracking

According to the above description, the liquid hydrocarbon product comprising C₄⁺ hydrocarbons may be obtained following a step of converting a synthesis gas intermediate via FT synthesis. For example, the liquid hydrocarbon product may be separated from the FT product, which, according to preferred embodiments, may correspond in quantity and in composition to the FT synthesis effluent. The liquid hydrocarbon product may be separated, more particularly, as a fraction of the FT synthesis effluent that is enriched in C₄⁺ hydrocarbons, using techniques known in the art (e.g., phase separation and/or fractionation). Optionally, in more particular embodiments according to the above description, the liquid hydrocarbon product may be obtained following a step of converting a synthesis gas intermediate via FT synthesis in combination with cracking. To the extent that the term “cracking” is used throughout this disclosure to describe additional reactions occurring in situ with FT synthesis, and/or downstream of FT synthesis, to reduce the molecular weight of C₂₀⁺ hydrocarbons produced, this term should be understood to encompass, in preferred embodiments, “cracking with isomerization,” in view of cracking catalysts described herein having activity for cracking, optionally together with isomerization, of these hydrocarbons, which reactions are both beneficial in terms of reducing and/or eliminating hydrocarbons that are solid at room temperature (i.e., wax) and increasing the branching in the molecular structure of liquid hydrocarbons. Likewise, the term “polishing” encompasses additional cracking reactions, as well as additional isomerization reactions, occurring in a separate cracking or polishing reactor, downstream of an FT reactor.

In the case of the liquid hydrocarbon product being obtained from a combination of FT synthesis and cracking, the latter reaction may be performed either in a downstream cracking reactor and/or possibly within the FT reactor itself, i.e., in situ. Therefore, the liquid hydrocarbon product, as a fraction of the FT synthesis effluent, may be more particularly a fraction of such FT synthesis effluent that is obtained as a product of the FT reactor with in situ cracking. As described above, cracking, whether in situ in the FT reactor or in a separate reactor downstream of the FT reactor, may be performed in combination with isomerization, for removing wax from the liquid hydrocarbon product (and in the case it is combined with isomerization, increasing branching in the molecular structure of the liquid hydrocarbon products), otherwise present in the FT synthesis effluent in the absence of such cracking (and optional isomerization). In this regard, any separate “cracking reactor,” positioned downstream of the FT reactor and used to condition (by reducing or eliminating wax from) the liquid hydrocarbon product in this manner prior to separation and/or recovery of C₄⁺ hydrocarbon fractions, may alternatively be referred to as a “polishing reactor.” Whether or not the FT synthesis effluent is obtained following the FT synthesis reaction alone or otherwise following the FT synthesis reaction in combination with in situ cracking (e.g., a combination of in situ cracking and in situ isomerization), the liquid hydrocarbon product may be a fraction of this FT synthesis effluent, obtained from the FT reactor, and may be separated therefrom. In the case of FT synthesis followed by cracking in a separate cracking or polishing reactor, the liquid hydrocarbon product may be a fraction of the polishing effluent. In this case of the FT synthesis effluent being obtained following cracking in a separate cracking or polishing reactor, the product immediately downstream of an FT reactor (e.g., withdrawn from this reactor) may be the FT synthesis effluent, which may correspond in quantity and in composition to the cracking feed, or feed to the cracking or polishing reactor (e.g., the FT synthesis effluent and cracking feed may be identical in the case of no intervening operations being performed between the FT reactor and the cracking or polishing reactor).

In the case of the FT synthesis effluent being obtained following the FT synthesis reaction in combination with in situ cracking, an “FT product” may be considered a hypothetical (or transient intermediate) product that comprises hydrocarbons present in the FT synthesis effluent but, unlike the FT synthesis effluent, further comprises hydrocarbons otherwise obtained from FT synthesis alone (in the absence of cracking), and especially a wax fraction (e.g., comprising normal C₂₀⁺ hydrocarbons) that, in the FT synthesis effluent, has been converted to normal or branched C₄-C₁₉ hydrocarbons, contributing to the yield of liquid hydrocarbons present in the FT synthesis effluent. The FT product may therefore correspond to an FT synthesis effluent, in the case of the FT reactor operating without in situ cracking.

As would be appreciated by those having skill in the art, with knowledge gained from the present disclosure, optional and additional separation and/or reaction (e.g., cracking) steps may be undertaken, respectively, to (i) increase the concentration of C₄⁺ hydrocarbons in the liquid hydrocarbon product and/or (ii) increase the yield of C₄⁺ hydrocarbons obtained from carbon in the gaseous feed mixture, and consequently the overall yield of the liquid hydrocarbon product. According to specific embodiments, hydrocarbon products or fractions (e.g., following separation from the FT synthesis effluent or polishing effluent) may comprise liquid hydrocarbons in a combined amount of at least about 60 wt-% (e.g., from about 60 wt-% to about 100 wt-%), at least about 90 wt-% (e.g., from about 90 wt-% to about 100 wt-%), or at least about 95 wt-% (e.g., from about 95 wt-% to about 99 wt-%). Together with such combined amounts, or alternatively, hydrocarbon products or fractions may comprise (i) hydrocarbons characteristic of naphtha or gasoline boiling-range hydrocarbons, such as C₄-C₉ hydrocarbons, (ii) hydrocarbons characteristic of jet fuel-boiling range hydrocarbons, such as C₉-C₁₆ hydrocarbons, and/or (ii) hydrocarbons characteristic of diesel boiling range hydrocarbons, such as C₁₆-C₂₅ hydrocarbons, in amount of at least about 25 wt-% (e.g., from about 25 wt-% to about wt-%), at least about 40 wt-% (e.g., from about 40 wt-% to about 80 wt-%), or at least about wt-% (e.g., from about 50 wt-% to about 75 wt-%). According to other specific embodiments (e.g., using recycle of an H₂/CO₂-enriched fraction or a hydrocarbon/CO₂ enriched fraction, in combination with cracking), at least about 40% (e.g., from about 40% to about 95%), at least about 55% (e.g., from about 55% to about 95%), or at least about 70% (e.g., from about 70% to about 95%), or at least about 80% (e.g., from about 80% to about 99%) of the feed carbon content of the gaseous feed mixture (e.g., the carbon content of CH₄ and/or CO₂ present in this mixture) forms liquid hydrocarbon products. These percentages are equivalently expressed in terms of wt-% or mol-%.

According to further embodiments, recycling of separated fractions of the liquid hydrocarbon product, such as any separated fraction enriched in particular C₄⁺ hydrocarbons, for example separated fractions enriched in (i) naphtha or gasoline boiling range hydrocarbons (i.e., “a naphtha boiling-range hydrocarbon fraction”), (ii) jet fuel boiling-range hydrocarbons (i.e., a “jet fuel boiling-range hydrocarbon fraction”), or (iii) diesel boiling-range hydrocarbons (i.e., a “diesel boiling-range hydrocarbon fraction”), may be used to vary the product slate of those C₄⁺ hydrocarbons recovered from the process, corresponding to the product yields of the process. In this regard, particular processes may be carried out, in which none of the liquid hydrocarbon product (e.g., substantially no C₄⁺ hydrocarbons in the liquid hydrocarbon product, such as those present in separated fractions) is recycled to the process, for example such that naphtha boiling-range hydrocarbons, jet fuel boiling-range hydrocarbons, and diesel boiling-range hydrocarbons are recovered or removed (e.g., as separated fractions) as outputs of the process. In this case, these recovered C₄⁺ hydrocarbons may comprise jet fuel boiling-range hydrocarbons in an amount of at least about 55 wt-%, with naphtha boiling-range hydrocarbons and diesel boiling-range hydrocarbons in combination representing all or substantially all of the balance. For example, the recovered C₄⁺ hydrocarbons may comprise at least about 40 wt-% of naphtha boiling-range hydrocarbons and diesel boiling-range hydrocarbons in combination. Other particular processes, however, may be carried out by recycling C₄⁺ hydrocarbons, such as in the case of recycling all or a portion of a given, separated fraction, in order to decrease or eliminate the yield of the types of hydrocarbons being recycled, in favor of other types of hydrocarbons. For example, compared to the yields of recovered C₄⁺ hydrocarbons as described above without liquid hydrocarbon recycle, in the case of naphtha boiling-range hydrocarbons in the liquid hydrocarbon product (e.g., present in a separated fraction) being recycled to the process, the recovered C₄⁺ hydrocarbons may comprise jet fuel boiling-range hydrocarbons in an amount of at least about 80 wt-%, with diesel boiling-range hydrocarbons representing all or substantially all of the balance. For example, the recovered C₄⁺ hydrocarbons may comprise at least about 15 wt-% of diesel boiling-range hydrocarbons.

More generally, any hydrocarbon fraction may be recycled in order to decrease the yield of that hydrocarbon fraction while increasing the yield of one or more other hydrocarbon fractions. For example, a process operating with a high yield of jet fuel boiling range hydrocarbons, if desired, may be implemented by recycling all or substantially all naphtha boiling-range hydrocarbons and diesel boiling-range hydrocarbons (e.g., present in separated fractions), while recovering and removing all or substantially all jet fuel boiling-range hydrocarbons from the process. This operational flexibility advantageously resides in the reforming/RWGS catalysts described herein, in terms of their surprising “robustness” for converting any unwanted hydrocarbon fractions that may be recycled to the first stage of the process. This differs from the characteristics of conventional reforming catalysts, which lack the ability to convert heavier hydrocarbon fractions, such as diesel boiling-range hydrocarbons, in a stable manner. Operation with the recycle of certain hydrocarbon fractions thereby provides a simple and straightforward strategy for managing (increasing or decreasing) the overall process selectivity for certain hydrocarbons.

Those skilled in the art, with knowledge gained from the present disclosure, will appreciate more generally the manner in which recycling various types of C₄⁺ hydrocarbons will impact the yields of these various types, and other types, of C₄⁺ hydrocarbons recovered from the process. Importantly, the use of cracking as described herein provides yet another mechanism by which hydrocarbon yields may be managed, in this case by reducing or eliminating C₂₀⁺ hydrocarbons. In some embodiments, for example, the recovered C₄⁺ hydrocarbons may consist of, or consist essentially of, any one or more of naphtha boiling-range hydrocarbons, jet fuel boiling-range hydrocarbons, and/or diesel boiling range hydrocarbons (e.g., without hydrocarbons having fewer than 4 carbon atoms or more than twenty carbon atoms), due to recycle of a hydrocarbon/CO₂-enriched fraction, in combination with cracking. In other embodiments, the recovered C₄⁺ hydrocarbons may consist of, or consist essentially of, jet fuel boiling-range hydrocarbons and/or diesel boiling range hydrocarbons (e.g., without hydrocarbons having fewer than 9 carbon atoms or more than twenty carbon atoms), due to recycle of a hydrocarbon/CO₂-enriched fraction as well as recycle of naphtha boiling-range hydrocarbons, in combination with cracking.

An optional downstream cracking or polishing reactor, separate from the FT reactor, may be desirable, as described above, in embodiments in which the FT synthesis effluent, downstream of an FT reactor, comprises a substantial amount (e.g., at least about 1 wt-%, at least about 5 wt-%, at least about 10 wt-%, or at least about 20 wt-%) of normal C₂₀⁺ hydrocarbons that may be cracked to increase the overall yield of C₄⁺ hydrocarbons (e.g., C₄-C₁₉ hydrocarbons) as the liquid hydrocarbon product. Such cracking or polishing reactor may be combined with in situ wax cracking in the FT reactor, as described above, in the event that even with in situ cracking, some wax remains in the FT synthesis effluent. A cracking or polishing feed to a downstream cracking or polishing reactor may comprise some or all of the FT synthesis effluent, optionally following one or more intervening operations such as cooling, heating, pressurizing, depressurizing, separation of one or more components, addition of one or more components, and/or reaction of one or more components. In view of the temperatures and pressures typically used in cracking or polishing reactor(s) of the FT synthesis stage, relative to those used in the FT reactor(s) of this stage, the FT synthesis effluent may be heated, prior to cracking or polishing, to a temperature suitable for a cracking or polishing reactor, as described herein. In some embodiments, this heating may be the only intervening operation to which the FT synthesis effluent is subjected, to provide the cracking or polishing feed. Alternatively, for even greater operational simplicity and efficiency, even this heating may be omitted, in view of the possibility for the FT reaction conditions to include a temperature that is the same or substantially the same as (e.g., within about 10° C. (18° F.) of) that used in the downstream cracking or polishing reactor, for example within a temperature range as described below with respect to the cracking or polishing reaction conditions. In other embodiments, intervening operations that may be omitted include pressurizing and depressurizing, as it has been discovered that cracking or polishing reaction conditions can advantageously include a same or substantially same pressure as described above with respect to FT reaction conditions. For example, a pressure in a cracking or polishing reactor can be the same pressure as in an upstream FT reactor, reduced by a nominal pressure drop associated with the piping and possibly other process equipment between these reactors. Therefore, costs for pressurization (compression) or depressurization (expansion) of the FT synthesis effluent, upstream of the cracking or polishing reactor, can be advantageously avoided. As with intervening operations between the reforming stage or RWGS stage and the FT synthesis stage, the use of no intervening operations, limited intervening operations, and/or the omission of certain intervening operations between an FT reactor and a cracking or polishing reactor of the FT synthesis stage results in advantages associated with the overall simplification of the integrated process. Particular advantages result, for example, if all or substantially all of the synthesis gas intermediate is used in the FT feed and/or all or substantially all of the FT synthesis effluent is used in the cracking or polishing feed. In other embodiments, all or substantially all of the synthesis gas intermediate, (e.g., except for a condensed water-containing portion), is used in the FT feed and/or all or substantially all of the FT synthesis effluent is used in the cracking or polishing feed.

Conditions in the cracking or polishing reactor(s) are therefore suitable for the conversion of normal C₂₀⁺ hydrocarbons, which are solid at room temperature, to additional hydrocarbons, and particularly C₄-C₁₉ hydrocarbons, which are liquid at room temperature. Insofar as cracking is generally carried out to convert a wax fraction of hydrocarbons obtained from FT synthesis, the terms (i) “wax cracking,” (ii) “wax cracking reactor,” (iii) “wax cracking conditions,” etc. may be substituted for, or used interchangeably as an alternative to, the respective terms (i) “cracking,” “polishing,” (ii) “cracking reactor,” “polishing reactor,” (iii) “cracking conditions,” “polishing conditions,” etc. as used herein. A cracking or polishing reactor may be incorporated into an FT reactor, for example by using a bed of cracking or polishing catalyst directly following, or at least downstream of, a bed of FT catalyst within a single vessel, or otherwise interspersing the two catalyst types within a single vessel. Alternatively or in combination, the use of at least one separate cracking or polishing reactor (e.g., as a separate cracking reactor vessel) is preferred, such that cracking reaction conditions can be maintained independently of FT reaction conditions as described above. A separate cracking or polishing reactor may be advantageous, for example, for (i) maintaining some or all of the cracking or polishing catalyst, used in the FT synthesis stage, in a different reactor type, compared to an FT reactor, such as maintaining the cracking or polishing catalyst in a fixed bed reactor that is normally simpler in design compared to an FT reactor, as a fixed bed reactor normally does involve not the same design constraints in terms of the ability to remove reaction heat, (ii) removing and/or replacing the cracking or polishing catalyst at times that do not necessarily coincide with (e.g., at differing intervals relative to) removing and/or replacing the FT catalyst, and/or (iii) operating the cracking or polishing reactor at a different temperature (e.g., at a higher temperature) or at other differing conditions compared to the FT reactor. The term “cracking catalyst” may be, but is not necessarily, reserved for catalysts having cracking and optionally isomerization activity, which are contained in situ in an FT reactor, whereas the term “polishing catalyst” may be, but is not necessarily, reserved for catalysts having cracking and/or isomerization activity, which are contained in a separate “polishing reactor,” downstream of the FT reactor. In the case of using both a “cracking catalyst” in situ in an FT reactor and one or more “polishing catalysts” in a separate polishing reactor, such a polishing catalyst may have the same composition as, and/or be in the same form as (e.g., in the form of spheres or cylinders), the cracking catalyst. More generally, any FT catalyst or cracking catalyst may independently have a form and/or dimensions as described above with respect to a reforming/RWGS catalyst. The polishing reactor may contain at least two catalysts, one having wax cracking activity (and optionally isomerization activity) and one having isomerization activity (and optionally wax cracking activity), or the polishing reactor contain one catalyst having activity for only wax cracking, only isomerization, or both wax cracking and isomerization. In the event that the FT reactor the polishing reactors have different designs and therefore present different reaction conditions to the contain catalysts, the cracking catalyst may have a different composition and/or form as any one of the one or more polishing catalysts having wax cracking activity.

With respect to the use of a separate cracking or polishing reactor, it may be important to maintain the FT synthesis effluent (or at least any portion of this effluent used in the cracking or polishing reactor), from the outlet (effluent) of the FT reactor to the inlet of the cracking or polishing reactor, at an elevated temperature to avoid condensation of liquid hydrocarbons and/or deposition of solid hydrocarbons, given the large distribution of carbon numbers of hydrocarbons produced from FT synthesis. Such condensation and/or deposition may be prevented if all or substantially all of the FT synthesis effluent is maintained in the vapor phase from the outlet of the FT reactor to the inlet of the cracking or polishing reactor. For example, the FT synthesis effluent may be maintained at a temperature of at least about 66° C. (150° F.), at least about 121° C. (250° F.), at least about 216° C. (420° F.), or even at least about 327° C. (620° F.), from the effluent of the FT reactor to the inlet of the cracking or polishing reactor, such as in the case of heating the FT synthesis effluent from this temperature to a temperature representative of cracking or polishing reaction conditions, as described herein. Such temperatures (suitable for use in at least one cracking or polishing reactor) may be in a range from about 200° C. (392° F.) to about 400° C. (752° F.), or from about 225° C. (437° F.) to about 300° C. (572° F.). Other cracking reaction conditions can include a gauge pressure from about 621 kPa (90 psig) to about 5.00 MPa (725 psig), or from about 2.50 MPa (362 psig) to about 3.50 MPa (508 psig).

In the cracking or polishing reactor(s), the cracking feed may be contacted with a suitable cracking or isomerization catalyst (e.g., bed of cracking catalyst particles disposed within the cracking reactor) under cracking or polishing reaction conditions, which may include a temperature and/or pressure described above. The cracking reactions may be more specifically hydrocracking reactions, which as understood in the art refer to reactions of hydrocarbons with hydrogen to produce hydrocarbons having a lower number of carbon atoms and consequently a lower molecular weight. Hydrocracking is beneficial for its overall impact on the carbon number distribution of the cracking or polishing feed, which may correspond to that of the FT product or FT synthesis effluent, and in particular for reducing the percentage by weight of, and possibly eliminating, normal C₂₀⁺ hydrocarbons present in cracking feed, which may correspond to those present in the FT product or FT synthesis effluent, in favor of C₄-C₁₉ hydrocarbons as components of the liquid hydrocarbon product. As described above, polishing may further include isomerization of normal C₂₀⁺ hydrocarbons to branched hydrocarbons that do not contribute to undesirable wax and isomerization of liquid hydrocarbons to branched hydrocarbons that improve the properties of the liquid hydrocarbons, and these isomerization reactions may be more specifically hydroisomerization reactions. In view of the cracking or polishing reactor being used to perform hydrocracking and hydroisomerization, this cracking or polishing reactor may be considered, more generally, a hydrotreating reactor, with the cracking or polishing catalysts contained in this reactor being considered, more generally, hydrotreating catalysts.

As hydrocracking and optionally hydroisomerization reactions require hydrogen, in some embodiments this hydrogen is present in the cracking or polishing feed and/or FT synthesis effluent (or portion thereof) that is input to the cracking or polishing reactor. For example, hydrogen in the synthesis gas intermediate that is unconverted in the downstream FT reactor may allow operation of the cracking or polishing reactor without the need for a supplemental source of hydrogen being added to the cracking or polishing reactor or downstream of the FT reactor. According to some embodiments, hydrogen is present in the cracking or polishing feed at a concentration of least about 5 mol-% (e.g., from about 5 mol-% to about 55 mol-%), at least about 10 mol-% (e.g., from about 10 mol-% to about 45 mol-%), or at least about 15 mol-% (e.g., from about 15 mol-% to about 35 mol-%), or at least about 20 mol-% (e.g., from about 20 mol-% to about 40 mol-%), without the introduction of a supplemental source of hydrogen, beyond the hydrogen produced in the reforming stage or RWGS stage and/or present in the synthesis gas intermediate. These concentrations of hydrogen may correspond to those present in the FT synthesis effluent. According to other embodiments, a supplemental source of hydrogen, added to a cracking or polishing reactor, or added upstream of such reactor (e.g., downstream of an FT reactor), may be used to achieve such hydrogen concentrations. A representative supplemental source of hydrogen is hydrogen that has been purified (e.g., by PSA or membrane separation) or hydrogen that is impure (e.g., syngas). A representative supplemental source of hydrogen may be one or more gaseous feed mixture components, such as a portion of one of these components that could otherwise be fed to the reforming stage or the RWGS stage, but that is instead fed directly to the polishing or cracking reactor and therefore does not actually contribute to the gaseous mixture. For example, the one or more gaseous feed mixture components may include one or more fresh gaseous feed mixture components and/or one or more recycle gaseous feed mixture components as described herein, and a portion of the one or more fresh gaseous feed mixture components may be fed directly to the polishing reactor. In more particular embodiments, the one or more fresh gaseous feed mixture components, the portion of which is fed directly to the at least one polishing reactor, is a fresh makeup H₂-containing feed comprising hydrogen, such as in an amount of at least about 50 mol-%, at least about 80 mol-%, at least about 95 mol-% hydrogen. Some or all of this hydrogen may be, more specifically, electrolysis hydrogen, fossil hydrogen with CCS, bio-gasification hydrogen, or methane pyrolysis hydrogen.

Suitable cracking/hydrocracking and/or isomerization/hydroisomerization reactions may also be performed in the presence of steam (H₂O) that may likewise, or alternatively, be present in the cracking or polishing feed and/or FT synthesis effluent, for example as a product of the RWGS reaction occurring in the upstream reforming stage or RWGS stage. For example, a supplemental source of steam may be added to a cracking or polishing reactor, or added upstream of such reactor (e.g., downstream of an FT reactor). According to some embodiments, steam may be present in the cracking or polishing feed and/or FT synthesis effluent at a concentration of least about 5 mol-% (e.g., from about 5 mol-% to about 45 mol-%), at least about 10 mol-% (e.g., from about 10 mol-% to about 40 mol-%), or at least about 15 mol-% (e.g., from about 15 mol-% to about 35 mol-%), with or without the introduction of a supplemental source of steam, beyond that produced in the reforming stage or RWGS stage and/or present in the synthesis gas intermediate.

Representative cracking or polishing catalysts comprise at least one cracking active metal on a solid support. The phrase “on a solid support” is intended to encompass catalysts in which the active metal(s) is/are on the support surface and/or within a porous internal structure of the support. Representative cracking or polishing active metals may be selected from Groups 12-14 of the Periodic Table, such as from Group 13 or Group 14 of the Periodic Table. A particular cracking or polishing active metal is gallium. The at least one cracking active metal may be present in an amount, for example, from about 0.1 wt-% to about 10 wt-%, from about 0.5 wt-% to about 8 wt-%, or from about 1 wt-% to about 5 wt-%, based on the weight of the cracking or polishing catalyst. If a combination of cracking active metals is used, such as a combination of metals selected from Groups 12-14 of the Periodic Table, then such metals may be present in a combined amount within these ranges. Generally, the cracking or polishing catalysts may comprise no metal(s) on the support in an amount, or combined amount, of greater than about 1 wt-%, or greater than about 0.5 wt-%, based on the weight of the cracking or polishing catalyst, other than the cracking or polishing active metal(s) described above (e.g., no metals other than metals of Groups 12-14 of the Periodic Table, no metals other than metals of Groups 13 or Group 14 of the Periodic Table, or no metals other than gallium, in this amount or combined amount). Preferably, the cracking or polishing catalyst comprises no metals on the support, other than the cracking or polishing active metal(s) described above (e.g., no metals other than metals of Groups 12-14 of the Periodic Table, no metals other than metals of Groups 13 or Group 14 of the Periodic Table, or no metals other than gallium).

In order to promote cracking activity, the solid support of the cracking or polishing catalyst may be more particularly a solid acidic support. The acidity of a support may be determined, for example, by temperature programmed desorption (TPD) of a quantity of ammonia, i.e., using NII₃-TPD as described above, with the analysis specifically being performed on an ammonia-saturated sample of the support, over a temperature from 275° C. (527° F.) to 500° C. (932° F.), which is beyond the temperature at which the ammonia is physisorbed. The quantity of acid sites, in units of millimoles of acid sites per gram (mmol/g) of support, therefore corresponds to the number of millimoles of ammonia that is desorbed per gram of support in this temperature range. A representative solid support comprises a zeolitic or non-zeolitic molecular sieve and has at least about 15 mmol/g (e.g., from about 15 to about 75 mmol/g) of acid sites, or at least about 25 mmol/g (e.g., from about 25 to about 65 mmol/g) of acid sites, measured by NH₃-TPD. In the case of zeolitic molecular sieves, acidity is a function of the silica to alumina (Si02/A1203) molar framework ratio, and, in embodiments in which the solid support comprises a zeolitic molecular sieve (zeolite), its silica to alumina molar framework ratio may be less than about 60 (e.g., from about 1 to about 60), or less than about 40 (e.g., from about 5 to about 40). Particular solid supports may comprise one or more zeolitic molecular sieves (zeolites) having a structure type selected from the group consisting of FAU, FER, MEL, MTW, MWW, MOR, BEA, LTL, MFI, LTA, EMT, ERI, MAZ, MEI, and TON, and preferably selected from one or more of FAU, FER, MWW, MOR, BEA, LTL, and MFI. The structures of zeolites having these and other structure types are described, and further references are provided, in Meier, W. M, et al., Atlas of Zeolite Structure Types, 4^{th} Ed., Elsevier: Boston (1996). Specific examples include zeolite Y (FAU structure), zeolite X (FAU structure), MCM-22 (MWW structure), and ZSM-5 (MFI structure), with ZSM-5 being exemplary.

Solid supports other than zeolitic and non-zeolitic molecular sieves include metal oxides, such as any one or more of silica, alumina, titania, zirconia, magnesium oxide, calcium oxide, strontium oxide, etc. In representative embodiments, the solid support may comprise (i) a single type of zeolitic molecular sieve, (ii) a single type of non-zeolitic molecular sieve, or (iii) a single type of metal oxide, wherein (i), (ii), or (iii) is present in an amount greater than about 75 wt-% (e.g., from about 75 wt-% to about 99.9 wt-%) or greater than about 90 wt-% (e.g., from about 90 wt-% to about 99 wt-%), based on the weight of the cracking or polishing catalyst. Other components of the support, such as binders and other additives, may be present in minor amounts, such as in an amount, or combined amount, of less than about 10 wt-% (e.g., from about 1 wt-% to about 10 wt-%), based on the weight of the cracking or polishing catalyst.

An exemplary cracking catalyst comprises gallium as the cracking active metal, present in an amount as described above (e.g., from about 0.5 wt-% to about 8 wt-%, such as from about 1 wt-% to about 5 wt-%, based on the weight of the cracking catalyst) on a support comprising, or possibly consisting essentially of, ZSM-5. Representative silica to alumina molar framework ratios of the ZSM-5 are described above.

Cracking or polishing catalysts and their associated reaction conditions described herein may generally be suitable for achieving a conversion of normal C₂₀⁺ hydrocarbons to C₁-C₁₉ hydrocarbons of at least about 50% (e.g., from about 50% to about 100%), at least about 70% (e.g., from about 70% to about 98% or from about 70% to about 100%), or at least about 90% (e.g., from about 90% to about 95% or from about 90% to about 100%). In the case of using both a cracking catalyst contained in an FT reactor for in situ cracking, as well as a polishing catalyst contained in a separate polishing reactor downstream of the FT reactor, these conversion levels may be representative of the conversion obtained from the combined cracking catalyst and polishing catalyst (e.g., as determined based on the amount of C₂₀₊ hydrocarbons remaining in the polishing effluent). Conversion of C₂₀⁺ hydrocarbons is important for improving the yield of C₄-C₁₉ hydrocarbons, and consequently the overall yield of the liquid hydrocarbon product, compared to the operation of the FT synthesis stage without in situ cracking or a separate polishing reactor. Preferably, in the case of a separate cracking or polishing reactor (whether or not used in combination with in situ cracking in an FT reactor), at least about 40% (e.g., from about 40% to about 100%), at least about 55% (e.g., from about 55% to about 98% or from about 55% to about 100%), or at least about 65% (e.g., from about 65% to about 97% or from about 65% to about 100%) of the C₂₀⁺ hydrocarbons in the FT synthesis effluent are converted to C₄-C₁₉ hydrocarbons. That is, the yields of these hydrocarbons from the conversion of normal C₂₀⁺ hydrocarbons in a separate cracking or polishing reactor are within these ranges. Preferably, the polishing effluent (product of the polishing reactor) comprises less than about 10 wt-%, or even less than about 5 wt-% of hydrocarbons that are solid at room temperature (e.g., normal C₂₀⁺ hydrocarbons). Advantageously, an FT synthesis stage utilizing in situ cracking and/or a separate polishing reactor can effectively provide a FT synthesis effluent and/or polishing effluent with no or substantially no residual wax, or a sufficiently low content of hydrocarbons that are solid at room temperature, such that these hydrocarbons remain solubilized in any recovered C₄⁺ hydrocarbons (e.g., a recovered diesel boiling-range hydrocarbon fraction) obtained as outputs of the process.

Embodiments of the invention are therefore directed to the use of a cracking or polishing reactor, following an FT reactor, to improve the overall selectivities to, and yields of, desired products and/or decrease the overall selectivities to, and yields of, undesired products, relative to the FT synthesis stage in the absence of cracking or polishing, i.e., relative to a baseline FT synthesis stage without in situ cracking or a separate polishing reactor. For example, in representative embodiments, the selectivity to, and/or yield of, of C₄-C₁₉ hydrocarbons may be increased by at least about 15% (e.g., from about 15% to about 70%), at least about 30% (e.g., from about 30% to about 65%), or at least about 45% (e.g., from about 45% to about 60%) in an FT synthesis stage utilizing in situ cracking and/or a separate polishing reactor, relative to the baseline FT synthesis stage. Selectivities to C₄-C₁₉ hydrocarbons are based on the percentage of carbon in CO converted by FT, which results in these hydrocarbons. Yields of C₄-C₁₉ hydrocarbons are based on the percentage of carbon in CO introduced to the FT synthesis stage (e.g., CO introduced with the FT feed, whether converted or unconverted), which results in these hydrocarbons. These increases in selectivity to, and/or yield of, C₄-C₁₉ hydrocarbons, as a result of incorporating in situ cracking and/or a separate polishing reactor, can be achieved without a significant difference between the CO conversion obtained in the baseline FT synthesis stage and that obtained in the FT synthesis stage utilizing in situ cracking and/or a separate polishing reactor. For example, the CO conversion values obtained in both cases may be within a range as described above with respect to the performance criteria of the FT synthesis stage. That is, the use of in situ cracking and/or a separate polishing reactor typically does not significantly impact the CO conversion obtained in the FT synthesis stage, such that the CO conversion achieved in both the baseline FT synthesis stage and FT synthesis stage utilizing in situ cracking and/or a separate polishing reactor may be the same or substantially the same. In representative embodiments, the per-pass selectivity to, and/or yield of, of C₄-C₁₉ hydrocarbons may be at least about 45% (e.g., from about 45% to about 85%), at least about 50% (e.g., from about 50% to about 80%), or at least about 55% (e.g., from about 55% to about 75%) in an FT synthesis stage utilizing in situ cracking and/or a separate polishing reactor.

The conversion levels in a cracking or polishing reactor, as described above, may be based on “per-pass” conversion, achieved in a single pass through the reactor, or otherwise based on overall conversion, achieved by returning a recycle portion of the polishing effluent back to the cracking or polishing reactor, as described above with respect to FT synthesis. A desired conversion of normal C₂₀⁺ hydrocarbons may be achieved by adjusting the cracking or polishing reaction conditions described above (e.g., cracking reaction temperature and/or pressure), and/or adjusting the weight hourly space velocity (WHSV), as defined above. The cracking or polishing reaction conditions may include a weight hourly space velocity (WHSV) generally from about 0.05 hr⁻¹ to about 35 hr⁻¹, typically from about 0.1 hr⁻¹ to about 20 hr⁻¹, and often from about 0.5 hr⁻¹ to about 10 hr⁻¹. The cracking or polishing reaction conditions may optionally include returning a recycle portion of the polishing effluent, exiting the cracking or polishing reactor, back to the FT synthesis effluent, for combining with the polishing feed, or otherwise back to the polishing reactor itself. Recycle operation allows for operation at relatively low “per-pass” conversion through the cracking or polishing reactor, while achieving a high overall conversion due to the recycle. Preferably, however, the cracking or polishing reaction conditions include little or even no polishing effluent recycle. For example, the cracking or polishing reaction conditions may include a weight ratio of recycled polishing effluent to cracking feed (i.e., a “recycle ratio”), with this recycled polishing effluent and FT synthesis effluent together providing a combined feed to the cracking or polishing reactor, corresponding to those recycle ratios above with respect to FT synthesis. Preferably, the recycle ratio may be 0, meaning that no polishing effluent recycle is used, such that the per-pass conversion is equal to the overall conversion. Advantageously, in the absence of polishing effluent recycle, utility costs are saved and the overall design of an integrated process is simplified.

Embodiments of the invention are therefore directed to a process for converting normal C₂₀⁺ hydrocarbons in a feed comprising C₄-C₁₉ hydrocarbons, such as an FT synthesis effluent or polishing feed as described above, which may comprise all or a portion of an FT product as described above. The feed comprising normal C₂₀⁺ hydrocarbons may comprise, for example, C₄-C₁₉ hydrocarbons in an amount of at least about 40 wt-% (e.g., from about 40 wt-% to about wt-%), or at least about 50 wt-% (e.g., from about 50 wt-% to about 80 wt-%), based on the weight of total hydrocarbons, or based on the weight of the feed. The feed may further comprise H₂ and/or H₂O (e.g., in amounts as described above with respect to an FT synthesis effluent), CO, and/or CO₂. The process comprises contacting the feed with a cracking catalyst as described above, for example comprising an active metal selected from Groups 12-14 of the Periodic Table (e.g., gallium) on a zeolitic molecular sieve support (e.g., ZSM-5), to achieve conversion of the normal C₂₀⁺ hydrocarbons at conversion levels, and with yields and selectivities to C₄-C₁₉ hydrocarbons, as described herein.

As described above, the step of converting the synthesis gas intermediate to the liquid hydrocarbon product, in the case of this step comprising FT synthesis in combination with cracking, may involve the use of an FT reactor, in which in situ, or integrated, cracking is performed. Examples of such a reactor include a single vessel that contains a fixed bed of cracking catalyst downstream of (e.g., directly following) a fixed bed of FT catalyst, or that otherwise contains a single fixed bed having the two catalysts interspersed or mixed in a suitable weight ratio. Alternatively, a fluidized bed of the two catalyst types as described herein may be used, with advantages of fluidized bed reactor operation residing in increased heat and mass transfer and therefore overall uniformity of FT and cracking reaction conditions. In alternative embodiments, two catalysts may be used in a slurry bed (e.g., slurry bubble) configuration, or these catalysts may otherwise be incorporated in tubes of a multi-tubular configuration. An ebullated bed reactor may also be used. Any of these reactor configurations, such as a slurry reactor, multi-tubular reactor, or ebullated bed reactor, may be characteristic of an FT reactor generally, whether or not this reactor contains a catalyst, or catalyst functional constituent, for performing in situ, or integrated, cracking. Forms of FT catalysts and cracking catalysts described herein may therefore be suitable for these various reactor configurations and include spherical forms having relatively small average diameters, such as from about 100 μm to about 1 mm, or from about 250 μm to about 750 μm, as well as other forms (e.g., cylindrical) as described above. In general, FT synthesis in combination with in situ, or integrated, cracking refers to the use of at least one FT reactor of the FT synthesis stage performing both of these reactions, at least to some extent. Representative conditions in such reactor may include any of the FT reaction conditions or the cracking reaction conditions described above, such as any of the ranges of temperature, pressure, and WHSV given with respect to these conditions.

A further embodiment for carrying out FT synthesis in combination with in situ, or integrated, cracking involves the use of a single catalyst composition, namely a bi-functional catalyst comprising both an FT-functional constituent and a cracking-functional constituent, with these constituents corresponding in isolation to an FT catalyst and a cracking catalyst as described above. For example, in the case of the cracking-functional constituent, this may comprise one or more cracking active metals selected from Groups 12-14 of the Periodic Table; the one or more cracking active metals may be deposited on a solid acidic support; and this solid acidic support may comprise a zeolitic molecular sieve having a silica to alumina molar framework ratio of less than about 50. Likewise, in the case of the FT-functional constituent, in particular embodiments, this constituent corresponds to the FT catalyst as described above.

When combined in a single catalyst composition, the functional constituents of a bi-functional catalyst may be present in equal or substantially equal weight ratios. For example, the (i) FT-functional constituent and (ii) cracking-functional constituent may be present in the bi-functional catalyst in a weight ratio of (i):(ii) of about 1:1. Generally, however, this weight ratio may vary, for example the weight ratio of (i):(ii) may be from about 10:1 to about 1:10, such as from about to about 1:5, or from about 3:1 to about 1:3. A representative bi-functional catalyst may therefore comprise (i) an FT functional constituent comprising one or both of (a) one or more FT active metals and (b) a solid support of an FT catalyst (e.g., comprising one or more metal oxides), as described above, and (ii) a cracking-functional constituent comprising one or both of (a) one or more cracking active metals and (b) a solid support of a cracking catalyst (e.g., a solid acidic support), as described above. It can be appreciated from the above description, including the weight ratios in which (i) and (ii) may be combined, that (a) and (b) of (i), as well as (a) and (b) of (ii), may be present in a bi-functional catalyst as a whole, in amounts that are less than those amounts in which they are present in their respective FT catalysts and cracking catalysts, as described above. For example, in the case of an FT-functional constituent of a bi-functional catalyst, such bi-functional catalyst as a whole may comprise transition metal(s) (i.e., one or more FT active metals, such as Co, as described above) in lower amount, such as in an amount of at least about 3 wt-% (e.g., from about 3 wt-% to about 30 wt-%), and typically at least about 5 wt-% (e.g., from about 5 wt-% to about 25 wt-%), based on the weight of the bi-functional catalyst. Likewise, in the case of a cracking-functional constituent of a bi-functional catalyst, such bi-functional catalyst as a whole may comprise metal(s) of Groups 12-14 of the Periodic Table (i.e., one or more cracking active metals as described above) in a lower amount, such as in an amount from about 0.03 wt-% to about 2 wt-%, or from about 0.1 wt-% to about 1 wt-%, based on the weight of the bi-functional catalyst.

Accordingly, the step of converting the synthesis gas intermediate comprising an H₂/CO mixture to the liquid hydrocarbon product, via FT synthesis in combination with cracking, may be performed in an FT reactor with the cracking carried out in situ, or being integrated. This step may comprise, more specifically, contacting the synthesis gas intermediate (or the H₂/CO mixture) with a bi-functional catalyst having an FT-functional constituent and a cracking-functional constituent.

### Once-Through and Recycle Operation/Exemplary Embodiment

Processes as described herein for producing a liquid hydrocarbon product may be carried out with (configured for) once-through operation, whereby the gaseous feed mixture is input and the liquid hydrocarbon product (optionally following separation from an FT synthesis effluent or from a polishing effluent, as described above) is withdrawn, without recycle of any portion of material obtained in the first or second reaction stages. In the case of once-through operation, the “gaseous feed mixture” and “fresh makeup feed” (which may be representative of a combination of more than one fresh gaseous feed component) are normally equivalent, and the conversion levels and product yields obtained from the process represent those of a single pass through the stages of reforming and/or RWGS and FT synthesis. As described above, certain aspects of the invention are associated with liquid hydrocarbon production processes that allow for the effective management/conversion of CO₂ that is present in a gaseous feed mixture or a fresh makeup feed, which can be improved through recycle operation. In particular, the recycle of CO₂ (e.g., present in a fraction enriched in (i) H₂ and CO₂ or (ii) the hydrogen source and CO₂, which may be separated from an FT synthesis effluent or from a polishing effluent), back to the first stage (e.g., a reforming stage, such as a reforming/RWGS stage), and/or back to the second, FT synthesis stage for further reaction, can promote its complete or essentially complete, overall conversion. For example, in representative embodiments in which recycle operation is used as described herein, an overall conversion of CO₂ present in a fresh makeup feed (e.g., having a composition as described above with respect to a “gaseous feed mixture,” and which may be representative of a combination of two or more fresh gaseous feed components) may be at least about 90%, at least about 95%, or even at least about 99%, with deviations from complete or 100% conversion resulting substantially, or at least in part, from CO₂ losses in a purge exiting the gaseous recycle loop that is used to control the accumulation of unwanted impurities in this loop. That is, according to some embodiments, CO₂ introduced to the process in the fresh makeup feed may be recycled substantially to extinction. In terms of fractions, as described above, which may be separated and/or recovered from the FT synthesis effluent, a fraction enriched in (i) H₂ and CO₂ or (ii) the hydrogen source and CO₂ may, for example, be recycled to the first stage (e.g., a reforming stage, such as a reforming/RWGS stage) and/or to the second, FT synthesis stage to attain important advantages as described herein. In some cases, only a recycle portion of fraction (i) or (ii) may be recycled to the first stage, or otherwise parts of a recycle portion of fraction (i) or (ii) may be recycled to the first and second stages. As further described herein, conversions and product yields may further be adjusted by recycling at least a portion of C₄⁺ hydrocarbons present in the liquid hydrocarbon product, for example by recycling all or a portion of a separated fraction enriched in naphtha boiling range hydrocarbons or hydrocarbons of another type. Recycle in this case is generally to the first stage, in which such recycled hydrocarbons can be reformed to increase the yield of the synthesis gas intermediate comprising the H₂/CO mixture.

An exemplary embodiment of a process 1 for producing a liquid hydrocarbon product and utilizing recycle is depicted in FIG. 1. As illustrated, gaseous feed mixture 6 is provided to reforming stage or RWGS stage 100, which may include one or more reforming/RWGS reactors for contacting gaseous feed mixture 6 with a reforming/RWGS catalyst and under reforming/RWGS conditions as described herein. Reactions occurring in reforming stage or RWGS stage 100 produce synthesis gas intermediate 8, which may be subjected to any one or more intervening operations as described herein. For example, water, such as in the form of condensed liquid water 9, may be separated from synthesis gas intermediate 8 to provide FT feed Optionally or in combination with this removal of condensed liquid water 9, a portion of fraction 14 of FT synthesis effluent 12, which may be a fraction enriched in (i) H₂ and CO₂ or (ii) the hydrogen source and CO₂, as described herein, may be added to synthesis gas intermediate 8 to provide FT feed 10.

Fraction 14 of FT synthesis effluent 12 may be, more particularly, a fraction enriched in (i) H₂ and CO₂, relative to liquid hydrocarbon product 16 and also relative to FT synthesis effluent 12. This fraction enriched in (i) may alternatively be referred to as an H₂/CO₂-enriched fraction and is generally a gaseous fraction (e.g., a substantially completely, or completely, vapor phase fraction), which may be used to form a gaseous recycle loop of the process. This fraction may comprise H₂ and CO₂ in a combined amount of at least about 20 mol-% (e.g., from about 20 mol-% to about 95 mol-%), at least about 40 mol-% (e.g., from about 40 mol-% to about 90 mol-%), or at least about 60 mol-% (e.g., from about 60 mol-% to about 85 mol-%). The balance of this fraction may comprise CO, CH₄, C₂H₆, C₃H₈,and/or H₂O. For example, the balance may comprise all, or substantially all, of one of these components, or otherwise two or more of these components. Fraction 14 of FT synthesis effluent 12 may be, more particularly, a fraction enriched in (ii) the hydrogen source and CO₂, relative to liquid hydrocarbon product 16 and also relative to FT synthesis effluent 12. This fraction enriched in (ii) may alternatively be referred to as a hydrocarbon/CO₂-enriched fraction (or methane/CO₂-enriched fraction) and is generally a gaseous fraction (e.g., a substantially completely, or completely, vapor phase fraction), which may be used to form a gaseous recycle loop of the process. This fraction may comprise hydrocarbons (e.g., one or more of CH₄, C₂H₆, C₃H₈) in a combined amount of at least about 20 mol-% (e.g., from about 20 mol-% to about 95 mol-%), at least about 40 mol-% (e.g., from about mol-% to about 90 mol-%), or at least about 60 mol-% (e.g., from about 60 mol-% to about mol-%). The balance of this fraction may comprise H₂, CO, and/or H₂O. For example, the balance may comprise all, or substantially all, of one of these components, or otherwise two or more of these components. Fraction 14 may therefore be considered a “light ends” fraction of FT synthesis effluent 12 (FIG. 1) or of polishing effluent 13 (FIG. 2), comprising hydrocarbons that are in the gas phase at room temperature (e.g., CH₄, C₂H₆, and/or C₃H₈) and that are generated as light hydrocarbon byproducts of FT synthesis and/or introduced in the fresh makeup feed as a hydrogen source.

Further according to the embodiment depicted in FIG. 1, second part 4b of fraction 14 of FT synthesis effluent 12, which may be a fraction enriched in (i) or (ii), may be added (or recycled) to synthesis gas intermediate 8, with the effect of altering the composition of FT feed 10, relative to that of synthesis gas intermediate 8, and more particularly with respect to the H₂:CO molar ratio of FT feed 10. Whether or not any intervening operations are performed, FT feed 10 (which in the absence of any intervening operation will be the same as synthesis gas intermediate 8), or a portion thereof, is provided to FT synthesis stage 200, which may include one or more FT reactors for contacting FT feed 10 (or synthesis gas intermediate 8) with an FT synthesis catalyst system and under FT reaction conditions as described herein. Reactions occurring in FT synthesis stage 200 produce FT synthesis effluent 12 that may be obtained directly from FT synthesis stage 200. All or a portion of FT synthesis effluent 12, optionally following a further intervening operation such as cooling via cooler 250, may be provided to separation stage 300 for separating various fractions as described above. According to the particular embodiment illustrated in FIG. 1, the separated fractions may include (e.g., among one or more other fractions), or may consist of, (A) a fraction 14 enriched in (i) or (ii) and (B) liquid hydrocarbon product 16 comprising C₄⁺ hydrocarbons and possibly having any of the more specific properties with respect to its composition, as described above. Depending on specific operations occurring in separation stage 300, the liquid hydrocarbon product may be obtained in the form of separated fractions 16b, 16c thereof, such as fraction 16b enriched in jet fuel boiling-range hydrocarbons and fraction 16c enriched in diesel boiling-range hydrocarbons. Alternatively, the liquid hydrocarbon product 16 may be separated downstream of separation stage, such as in separate liquid product separation stage 400, which may utilize a fractionator (e.g., distillation column) to resolve fraction 16b enriched in jet fuel boiling-range hydrocarbons and fraction 16c enriched in diesel boiling-range hydrocarbons. In addition to being enriched in these respective types of hydrocarbons, the separated fractions may more particularly consist of, or consist essentially of, these respective types of hydrocarbons. For example, separated fraction 16b may consist of, or consist essentially of, jet fuel boiling-range hydrocarbons, and separated fraction 16c may consist of, or consist essentially of, diesel boiling range hydrocarbons. A further example of a separated fraction is separated fraction 16a shown in FIG. 2, which may be enriched in, or more particularly may consist of, or consist essentially of, naphtha boiling-range hydrocarbons. According to the embodiment illustrated in FIG. 2, at least a portion of this separated fraction 16a is recycled to reforming stage or RWGS stage 100, although in other embodiments, such separated fraction may be completely recovered as an output of the process and thereby contribute to the yield of naphtha boiling-range hydrocarbons.

To improve overall CO₂ conversion and management, at least a portion of fraction 14 enriched in (i) or (ii) may be recycled back to upstream operations or stages of the process, including reforming stage or RWGS stage 100, and/or FT synthesis stage 200. Typically, for example, a recycle portion 4 of fraction 14 (e.g., a fraction enriched in (i) as described above, which may alternatively be referred to as an H₂/CO₂-enriched fraction, or a fraction enriched in (ii) as described above, which may alternatively be referred to as a hydrocarbon/CO₂-enriched fraction), may be obtained following the removal of purge 20 that serves to limit the accumulation of unwanted impurities in the gaseous recycle loop, and particularly non-condensable impurities such as N2 and others that may be present in fresh makeup feed 2. The separation of purge 20 provides recycle portion 4 of the H₂/CO₂-enriched fraction 14, which recycle portion 4 may then be advantageously utilized to improve performance of the overall process in various respects. For example, recycle portion 4 may be recycled to either or both stages 100, 200 to increase overall CO₂ conversion of the process (e.g., beyond a “per-pass” or once-through CO₂ conversion that may be obtained on the basis of either stage operating alone, or on the basis of both stages operating together). Alternatively, or in combination, CO₂ present in fraction 14 or recycle portion 4 thereof, may, when introduced to one or both stages 100, 200, and particularly FT synthesis stage 200, beneficially suppress or reduce a net CO₂ production in that stage (e.g., due to the water-gas shift reaction). According to the particular embodiment shown in FIG. 1, a first part 4a of recycle portion 4, following optional compression by compressor 360 in the case that stage 100 requires a higher pressure feed gas than is provided by recycle portion 4, may be recycled to reforming stage or RWGS stage 100 (e.g., by being combined with fresh makeup feed 2), and/or a second part 4b, following compression by compressor 350, may be recycled to FT synthesis stage 200 (e.g., by being combined with synthesis gas intermediate 8 or FT feed 10). In general, parts 4a, 4b, as well as purge 20 and recycle portion 4, will have the same composition as fraction 14, although in some embodiments this may not necessarily be the case (e.g., if purge 20 provided as a result of a separation that enriches this stream in certain unwanted impurities).

The selection of a given recycle configuration, in terms of recycling fraction 14 or any portion(s) thereof to certain stage(s) of the process, may depend at least in part on the above considerations with respect to increasing overall CO₂ conversion of the process and/or suppressing CO₂ production in a given stage. Having knowledge of the present disclosure, the skilled person would appreciate the applicability of these and other considerations to a given process within the scope of invention. As is apparent from the above description, the recycle portion 4 as well as any parts 4a, 4b thereof that may be routed to different locations all constitute “a portion of the fraction” 14, or alternatively “a part of the fraction” 14, with this fraction being enriched in (i) or (ii) as described throughout the present disclosure. Therefore, for example, gaseous feed mixture 6 may be provided to reforming stage or RWGS stage 100 as a combination of fresh makeup feed 2 and a portion of fraction 14 (e.g., all of recycle portion 4, or part 4a of this portion).

According to particular embodiments, fresh makeup feed 2 may comprise, or consist essentially of, biogas. In such embodiments, gaseous feed mixture 6 may comprise biogas that is present therein as a fresh makeup feed, or fresh gaseous mixture feed mixture component 2a, 2b (FIG. 2). According to other particular embodiments, fresh makeup feed 2 may comprise CO₂ that has been removed from the atmosphere (e.g., via direct air capture) or is captured from the exhaust stream of biomass combustion, and may further comprise electrolysis H₂ (e.g., generated via using renewable electricity), fossil hydrogen with CCS, bio-gasification hydrogen, or methane pyrolysis hydrogen. According to yet other particular embodiments, fresh makeup feed 2 may comprise the effluent from the gasification of biomass, including, for example, CO₂, H₂, and CO, and optionally CH₄, and may further comprise supplemental H₂ that is electrolysis H₂ (e.g., generated via using renewable electricity), fossil hydrogen with CCS, bio-gasification hydrogen, or methane pyrolysis hydrogen. According to yet other particular embodiments, fresh makeup feed 2 may comprise the stranded gases including, for example, CO₂, H₂, CH₄, and CO.

A further exemplary embodiment of a process 1 for producing a liquid hydrocarbon product is depicted in FIG. 2. In comparing FIG. 1 and FIG. 2, various aspects relating to processes described herein are apparent, including the ability to input recycle streams directly into reactors, as opposed to combining recycle streams with other process streams, prior to feeding the combined streams to these reactors. Therefore, for example, gaseous feed mixture 6 can result from the combination of first part 4a of fraction 14 with fresh makeup feed 2, as illustrated in FIG. 1, or otherwise may result in situ in a reactor of reforming stage or RWGS stage 100, from the combination of first part 4a of fraction 14 with fresh makeup feed, which, according to FIG. 2 is provided as a combination of fresh gaseous feed mixture components, namely fresh makeup CO₂- and/or CH₄-containing feed 2a and fresh makeup Hz-containing feed 2b, wherein feeds 2a and 2b may in certain embodiments consist of a single combined feed. In the same manner, FT feed 10, as illustrated in FIG. 1, may result in situ in FT reactor 200a, as illustrated in FIG. 2, from the combination of second part 4b of fraction 14 with synthesis gas intermediate 8. FIG. 2 further illustrates options for compression, such as through the use of synthesis gas intermediate/FT feed compressor 125 in addition to recycle gas compressors 350 and 360, with compressor 125 being used to obtain a suitable pressure in FT reactor 200a, consistent with FT reaction conditions described herein. FIG. 2 yet further illustrates options for water removal, such as using liquid product separation stage 400 for this purpose of removing condensed liquid water 9, optionally in combination with its removal from synthesis gas intermediate 8 as illustrated in FIG. 1.

As further illustrated in FIG. 2, FT synthesis stage 200 may, according to particular embodiments, comprise both FT reactor 200a and downstream polishing reactor 200b. Generally, in some embodiments consistent with this figure, the FT synthesis stage may comprise at least one FT reactor 200a containing a mixture of an FT catalyst and a cracking catalyst (such as those catalysts having compositions as described herein). The FT synthesis stage may further comprise at least one polishing reactor 200b downstream of the at least one FT reactor 200a, such as in the case of FT reactor 200a providing FT synthesis effluent 12, and the process further comprising feeding FT synthesis effluent 12 to polishing reactor 200b. The polishing reactor may contain one or more polishing catalysts, with the FT catalyst being completely or substantially absent in polishing reactor 200b. This FT catalyst, which may be completely or substantially absent in the polishing reactor, can refer to the specific FT catalyst contained in FT reactor 200a or can refer to any other FT catalyst as described herein. The polishing catalyst may have the same composition and/or the same form as the cracking catalyst contained in the FT reactor, or otherwise, depending on how the FT reactor and polishing reactor are operated, these compositions and/or forms may differ. Generally, in other embodiments consistent with FIG. 2, the FT synthesis stage may comprise at least one FT reactor containing a bi-functional catalyst having an FT-functional constituent and a cracking-functional constituent (such as those functional constituents having compositions as described herein). The FT synthesis stage may further comprise at least one polishing reactor 200b downstream of the at least one FT reactor 200a such as in the case of FT reactor 200a providing FT synthesis effluent 12, and the process further comprising feeding FT synthesis effluent 12 to polishing reactor 200b. The polishing reactor may contain a polishing catalyst, with the FT-functional constituent being substantially absent in the polishing catalyst. This FT-functional constituent, which may be completely or substantially absent in the polishing catalyst, can refer to the specific FT-functional constituent of catalyst contained in FT reactor 200a or can refer to any other FT-functional constituent as described herein. The polishing catalyst may have the same composition as the cracking-functional constituent, corresponding to the same composition as the bi-functional catalyst contained in the FT reactor, but excluding the FT-functional constituent. Otherwise, depending on how the FT reactor and polishing reactor are operated, the compositions of the polishing catalyst and cracking-functional constituent may differ.

Generally, in some embodiments consistent with FIG. 2, in the FT synthesis stage, the step of converting the synthesis gas intermediate 8, comprising the H₂/CO mixture, may comprise contacting this intermediate or this mixture with a mixture of an FT catalyst and a cracking catalyst (e.g., contained in FT reactor 200a), to provide FT synthesis effluent 12. Converting the synthesis gas intermediate 8, comprising the H₂/CO mixture, may further comprise contacting FT synthesis effluent 12 with a polishing catalyst (e.g., contained in polishing reactor 200b), for example in the substantial absence of the FT catalyst, to provide polishing effluent 13. Generally, in other embodiments consistent with FIG. 2, in the FT synthesis stage, the step of converting the synthesis gas intermediate 8, comprising the H₂/CO mixture, may comprise contacting this intermediate or this mixture with a bi-functional catalyst (e.g., contained in FT reactor 200a) having an FT-functional constituent and a cracking-functional constituent, to provide FT synthesis effluent 12. Converting the synthesis gas intermediate 8, comprising the H₂/CO mixture, may further comprise contacting FT synthesis effluent 12 with a polishing catalyst (e.g., contained in polishing reactor 200b), for example in which the FT-functional constituent is substantially absent, to provide polishing effluent 13. In any of such embodiments, the cracking catalyst or cracking-functional constituent, as the case may be, may have any composition as described herein, for example, such catalyst or functional constituent may comprise one or more cracking active metals selected from Groups 12-14 of the Periodic Table.

From the embodiment illustrated in FIG. 2, it can be appreciated that the gaseous feed mixture

may comprise, as components, fresh gaseous feed mixture components, as inputs to the process, as well as recycle gaseous feed mixture components. For example, with respect to fresh makeup feed 2 as shown in FIG. 1, this may be provided as a combination of fresh gaseous feed mixture components as shown in FIG. 2, namely fresh makeup CO₂- and/or CH₄-containing feed 2a and fresh makeup Hz-containing feed 2b, as described herein. All or a portion, such as first portion of fresh makeup Hz-containing feed 2b may be added directly to reforming stage or RWGS stage 100 (e.g., a reforming/RWGS reactor used in this stage) and may therefore be a component of the gaseous feed mixture. All or a portion, such as second portion 25b, of fresh makeup H₂-containing feed 2b may be added directly to polishing reactor 200b, as a polishing reactor co-feed. Therefore, in embodiments consistent with FIG. 2, in the FT synthesis stage, the polishing catalyst (e.g., contained in polishing reactor 200b) may be contacted with FT synthesis effluent 12 in addition to a fresh makeup H₂-containing feed 2b or a portion 25b thereof, having a composition as described herein. This feed may comprise, for example, H₂ derived from electrolysis of water, fossil hydrogen with CCS, bio-gasification hydrogen, or methane pyrolysis hydrogen. Regardless of the source, H₂ may be present in the fresh makeup H₂-containing feed 2b, and consequently in portions 25a, 25b having the same composition, in an amount of at least about 50 mol-% H₂.

Accordingly, the gaseous feed mixture may comprise first portion 25a of fresh makeup H₂-containing feed 2b, namely that portion being fed directly to reforming stage or RWGS stage 100 (e.g., a reforming/RWGS reactor used in this stage), which provides an input to the process. Representative processes may further comprise feeding second portion 25b of fresh makeup H₂-containing feed 2b directly to FT synthesis stage 200, or, in preferred embodiments, directly to polishing reactor 200b used in this stage. In this regard, aspects of the invention are associated with the discovery that feeding hydrogen, with a convenient source of this hydrogen being present in a fresh makeup H₂-containing feed 2b as described herein, directly to polishing reactor 200b can be beneficial in terms of promoting the hydrotreating (e.g., hydrocracking and hydroisomerization) reactions in this reactor. Importantly, directly introducing a portion 25b of this feed, which would otherwise be fed with portion 25a to reforming stage or RWGS stage 100, to polishing reactor 200b, reduces the CO partial pressure in this reactor, thereby improving performance of a polishing catalyst contained in this reactor, for converting C₂₀⁺ hydrocarbons, and/or otherwise improving stability of this catalyst (i.e., reducing the rate of its deactivation). As described above, portion 25b being fed directly to polishing reactor 200b may be, more particularly, a second portion, with a first portion 25a being fed directly to reforming stage or RWGS stage 100 (e.g., a reforming/RWGS reactor used in this stage).

Other particular aspects of the invention are associated with the realization that flows of fresh and recycle streams in processes described herein can be beneficially adjusted as a basis for controlling relevant processes parameters, including the CO concentration (CO mol-%) or CO partial pressure, and the H₂:CO molar ratio at various points (measurement or control points) in the FT synthesis stage (e.g., controlling CO mol-% or CO partial pressure in polishing reactor 200b and/or controlling H₂:CO molar ratio in FT reactor 200a). In certain exemplary embodiments relating to CO concentration (CO mol-%) control, a feed rate of fresh makeup H₂-containing feed (e.g., a feed rate of second portion 25b) to polishing reactor 200b is adjusted to maintain a CO mol-% or CO partial pressure in the FT synthesis stage, such as in polishing reactor 200b of this stage. For example, this feed rate may be adjusted to maintain a set point or maximum CO mol-% or CO partial pressure, which, in the case of polishing reactor 200b, may be a measured CO mol-% or CO partial pressure, or otherwise a calculated CO mol-% or CO partial pressure (e.g., based on other parameters indicative of CO mol-% or CO partial pressure) at the reactor inlet or upstream of the reactor (e.g., in the FT synthesis effluent or polishing feed). The feed rate may be increased, for example, in response to a measured or calculated CO mol-% or CO partial pressure that is above the set point or maximum, and conversely decreased in response to a measured or calculated CO mol-% or CO partial pressure that is below the set point or maximum. The adjustment may also include suspending flow (i.e., adjusting the feed rate to zero) of the fresh makeup H₂-containing feed and subsequently resuming flow. A set point or maximum CO mol-% in the total gaseous feed into stage 200b (comprising the combination of 12 and 25b) may be, for example, any discrete value from about 5 mol-% to about 25 mol-%, from about 3 mol-% to about 15 mol-%, or from about 1 mol-% to about 10 mol-%. A set point or maximum CO partial pressure may be, for example, any discrete value from about 34 kPa (5 psi) to about 1.38 MPa (200 psi), from about 34 kPa (5 psi) to about 689 kPa (100 psi), or from about 69 kPa (10 psi) to about 344 kPa (50 psi). As described herein, according to preferred embodiments, the fresh makeup H₂-containing feed, such as second portion 25b, may be fed directly to polishing reactor 200b, positioned downstream of FT reactor 200a.

Another process parameter that may be controlled in the FT synthesis stage, alternatively to, or in combination with, CO mol-% control or CO partial pressure control, is the H₂:CO molar ratio, which may be particularly significant from an operational standpoint in an FT reactor of this stage. As described herein, representative processes may comprise: (a) feeding, to the FT synthesis stage, at least a part of a fraction enriched in (i) H₂ and CO₂ or (ii) the hydrogen source and CO₂, with this fraction having been separated from an effluent of the FT synthesis stage; and (b) feeding, to the reforming and/or RWGS stage, at least a part of a fraction enriched in (i) H₂ and CO₂ or (ii) the hydrogen source and CO₂, with this fraction having been separated from an effluent of the FT synthesis stage. For example, second part 4b of fraction 14 may be fed to FT reactor 200a and first part 4a of fraction 14 may be fed to reforming and/or RWGS reaction 100. The fraction 4b fed to an FT reactor thereby provides at least a portion of the feed to this reactor (e.g., as FT feed 10 upstream of an FT reactor, as illustrated in FIG. 1, or in situ in FT reactor 200a, as illustrated in FIG. 2). The effluent of the FT synthesis stage, from which the fraction enriched in (i) or (ii) is separated, may be, more particularly, FT synthesis effluent 12 as illustrated in FIG. 1 or polishing effluent 13 as illustrated in FIG. 2. The feed rate of this fraction 4b enriched in (i) or (ii), may be adjusted to contribute to maintaining a H₂:CO molar ratio within a certain range in the FT synthesis stage, or, more particularly, in FT feed 10 as illustrated in FIG. 1 or in FT reactor 200a as illustrated in FIG. 2. Similarly, the fraction 4a, fed to a reforming/RWGS reactor provides at least a portion of the feed to this reactor (e.g., as a portion of feed 6 in FIG. 1 or along with feeds 2a and 25a in FIG. 2.). The feed rate of this fraction 4a enriched in (i) or (ii), may be adjusted to change the stoichiometry of the feed into the reforming and/or RWGS stage which may in turn change the H₂:CO ratio of the reforming and/or RWGS stage effluent, which in turn feeds the FT synthesis stage and thereby may contribute to maintaining a setpoint or minimum H₂:CO molar ratio in the FT synthesis stage. Such setpoint or minimum H₂:CO molar ratio may be a measured or calculated value (e.g., calculated based on a measured composition of the FT feed), which setpoint or minimum H₂:CO molar ratio may be any discreet value within ranges described herein with respect to the synthesis gas intermediate or FT feed. For example, the setpoint or minimum H₂:CO molar ratio may be any discreet value within a range from about 2.1 to about 2.5. Depending on the composition (e.g., hydrogen content) of the fraction enriched in (i) or (ii), the feed rates of the further fractions 4b and 4a may be adjusted, for example, in response to a measured or calculated H₂:CO molar ratio that is outside of a target range. The adjustment may also include suspending flow (i.e., adjusting the feed rate to zero) of this fraction, or part of this fraction, and subsequently resuming flow. The adjustment may be based on increasing or decreasing a flow rate, or otherwise increasing or decreasing a percentage, represented by part of the fraction being fed to the FT synthesis stage (e.g., FT reactor 200a of this stage), in relation to the entire fraction. As described herein, the part of fraction 14 enriched in (i) or (ii), and fed to the FT synthesis stage may be second part 4b. For example, the gaseous feed mixture may comprise first part 4a, as a recycle gaseous feed mixture component, which is fed to reforming stage or RWGS stage 100 (e.g., a reforming/RWGS reactor of this stage).

With respect to separating and/or recovering the liquid hydrocarbon product obtained from the FT synthesis stage, the effluent of this stage that contains this product may be, according to particular embodiments, FT synthesis effluent 12 as illustrated in FIG. 1 or polishing effluent 13 as illustrated in FIG. 2. Insofar as polishing effluent 13 may be downstream of FT synthesis effluent 12, separating the liquid hydrocarbon product from the polishing effluent can likewise include separating it from the FT synthesis effluent. According to representative processes, therefore, the liquid hydrocarbon product may be contained in the FT synthesis effluent (e.g., the effluent of FT reactor 200a) or the polishing effluent, and such processes may comprise separating the liquid hydrocarbon product comprising C₄⁺ hydrocarbons from the respective FT synthesis effluent or polishing effluent. In addition, a fraction enriched in (i) H₂ and CO₂ or (ii) the hydrogen source and CO₂, as described herein, may likewise be separated from the respective FT synthesis effluent or polishing effluent. For example, FIG. 1 illustrates the use of separation stage 300 to perform these separations (e.g., vapor/liquid separations) on FT synthesis effluent 12, whereas FIG. 2 illustrates the use of separation stage 300 in an analogous manner to perform these separations on polishing effluent 13. In either case, separation stage 300 may result in providing liquid hydrocarbon product 16 comprising, consisting of, or consisting essentially of, C₄⁺ hydrocarbons that are liquid at room temperature, and this product may then be further separated (e.g., fractionated) in liquid product separation stage 400. In the same manner as described herein with respect to recycling the fraction enriched in (i) or (ii) and separated from FT synthesis effluent 12, as illustrated in FIG. 1, these fractions may likewise be recycled when separated from polishing effluent 13, as illustrated in FIG. 2. For example, representative processes may further comprise recycling this fraction to the reforming stage or the RWGS stage 100 (e.g., to a reforming/RWGS reactor of this stage) and/or recycling it to the FT synthesis stage (e.g., to an FT reactor of this stage), and preferably to both stages. In some embodiments, the fraction enriched in (ii) the hydrogen source and CO₂, can be predominantly CH₄, originating from the hydrogen source being input to the process (e.g., in a fresh gaseous feed mixture component, such as a fresh makeup CO₂- and/or CH₄-containing feed 2a) and/or generated as a light hydrocarbon byproduct of FT synthesis, which becomes a component of the “light ends” fraction (ii) of the FT synthesis effluent. This fraction may comprise other light hydrocarbons, such as C₂H₆ and/or C₃H₈.

As illustrated in both FIGS. 1 and 2, processes may further comprise fractionating liquid hydrocarbon product 16, for example obtained from separation stage 300, into one or more separated fractions enriched in types of hydrocarbons. For example, whereas both FIGS. 1 and 2 illustrate separated fraction 16b enriched in jet fuel boiling-range hydrocarbons (which may be referred to as a jet fuel boiling-range fraction) and separated fraction 16c enriched in diesel fuel boiling-range hydrocarbons (which may be referred to as a diesel boiling-range fraction), FIG. 2 additionally illustrates separated fraction 16a enriched in naphtha boiling-range hydrocarbons (which may be referred to as a naphtha boiling-range fraction). To the extent that these separated fractions are recovered from the process as outputs, they may contribute to the yield of C₄⁺ hydrocarbons and represent all or substantially all C₄⁺ hydrocarbons present in the liquid hydrocarbon product, possibly with the exception of residual amounts of these hydrocarbons that may be present in condensed liquid water 9. As further described herein, representative processes may comprise recycling all or a portion of a separated fraction, thereby adjusting the product slate, in terms of proportions of the types of hydrocarbons recovered. For example, as illustrated in FIG. 2, at least a portion, such as hydrocarbon recycle 30b, of naphtha boiling-range fraction 16a, may be recycled to reforming stage or RWGS stage 100. In the case of only a portion being recycled, recovered portion 30a may represent hydrocarbons recovered from the process that contribute to the yield of C₄⁺ hydrocarbons.

The following examples are set forth as representative of the present invention. These examples are not to be construed as limiting the scope of the invention as other equivalent embodiments will be apparent in view of the present disclosure and appended claims.

### EXAMPLE 1

### Reforming of a Gaseous Feed Mixture

Pilot plant scale experiments were performed in which gaseous mixtures were fed continuously to a reactor containing catalyst particles having a composition of 1 wt-% Pt and 1 wt-% Rh on a cerium oxide support. The performance of the system for reforming/RWGS was tested at conditions of 0.9 hr⁻¹ WHSV, 864° C. (1587° F.), and a gauge pressure of 346 kPa (50 psig). The gaseous mixture tested was a composition (“IH² type feed”) containing methane, ethane, propane, and CO₂, in addition to H₂O, and simulating that obtained from the combined hydropyrolysis and hydroconversion of biomass, followed by removal of the bulk of the hydrogen via pressure swing adsorption (PSA). This gaseous feed mixture and the synthesis gas product (“IH² type product”) obtained from this feed, are summarized in Table 1 below.

TABLE 1

| | IH² Type | IH² Type |
|---|---|---|
| | Feed | Product |
| methane, mol-% | 11.1 | 0.3 |
| ethane, mol-% | 9.1 | 0 |
| propane, mol-% | 3.5 | 0 |
| CO₂, mol-% | 2.5 | 10.6 |
| water, mol-% | 49.5 | 12.7 |
| H₂, mol-% | 12.6 | 51.3 |
| CO, mol-% | 11.6 | 25.1 |
| % hydrocarbon | | 89 |
| conversion | | |
| H₂:CO molar ratio | | 2.37 |
| gas flow rate, cc/min | 800 | 2690 |

From these results, it can be seen that the CO₂-steam reforming catalyst and process can provide a synthesis gas product representing a high hydrocarbon conversion and having a H₂:CO molar ratio suitable for subsequent, direct processing via the Fischer-Tropsch reaction (e.g., without a prior (upstream) adjustment of this ratio).

### EXAMPLE 2

### Fischer-Tropsch (FT) Testing

FT testing was performed in an 0.5 inch (6 mm) water-jacketed upflow reactor with a mixture of 35% hydrocracking/isomerization catalyst (1% Ga-ZSM-5) and 65% Fischer-Tropsch Catalyst (20% Co-1% Pt, 1%Rh) followed by a fixed bed polishing reactor with 100% Ga-ZSM-5 catalyst. All the catalyst was 35-60 mesh size. The liquid product obtained was condensed and removed. The gas, separated from this product, continued to a duplicate FT+polishing reactor system and additional liquid product was condensed and removed. In this manner, each of the two reactor stages could attain 50-60% conversion, with a final overall conversion, through the two reactor stages, being well over 65% after sufficient time for the system to reach steady state. The gas separated from the second stage gas was then measured and analyzed. Even following more than 500 hours of testing, with intermittent shutdowns and restarting with hydrogen treatment, the Fischer-Tropsch catalyst had minimal deactivation. Several gallons of Fisher Tropsch liquid was collected during the testing period.

The FT system utilized an upflow, liquid-filled reactor, with gas bubbling through it. This reactor had good heat transfer due to the liquid, and good temperature uniformity due to the water jacket and the diluted mixed catalyst, which dissipated the reaction heat. Results from the FT testing over three testing periods are provided in Table 2.

TABLE 2

| | Test #1 | Test #2 | Test #3 |
|---|---|---|---|
| CONDITIONS | | | |
| feed flow rate cc/min | 2200 | 2200 | 2200 |
| feed rate, g/hr. | 59.6 | 60.0 | 59.6 |
| temperature, ° C. FT Rx(avg) | 207 | 207 | 207 |
| temperature, ° C. HC/HI Rx(avg) | 243 | 243 | 243 |
| pressure, psig | 450 | 470 | 470 |
| WHSV FT catalyst(total) | .42 | .42 | .42 |
| WHSV HC/HI catalyst(total) | .35 | .35 | .35 |
| FEED COMPOSITION | | | |
| feed mole % H₂ | 70 | 69.1 | 70 |
| feed mole % CO | 27.2 | 28.1 | 27.2 |
| feed mole % CH₄ | .9 | .9 | .9 |
| feed mole % CO₂ | 8.2 | 1.8 | 8.2 |
| H₂:CO molar ratio | 2.57 | 2.45 | 2.57 |
| RESULTS | | | |
| wt-% recovery | 97.5 | 95.2 | 96.9 |
| gas product cc/min | 850 | 670 | 850 |
| wt-% CO conversion | 73.4 | 80.6 | 73.0 |
| g/hr HC liquid (normalized) | 16.2 | 16.9 | 15.7 |
| g/hr wax(normalized) | 0 | .9 | 0 |
| | (trace) | | |
| g/hr water (normalized) | 19.7 | 20.8 | 19.8 |
| g/hr to gas (normalized) | 39.2 | 21.4 | 39.6 |
| wt-% to wax | 0 | 1.5 | 0 |
| % C Selectivity to liquid % | 83.2 | 79.3 | 82.7 |
| % C Selectivity to gas % | 16.8 | 16.7 | 17.2 |
| % C Selectivity to wax % | 0 | 3.9 | 0 |

The FT product was a high-quality, water-white hydrocarbon liquid product having less than 0.6 wt-% oxygen content and being easily separated from the water. A representative distillation curve for this product is shown in FIG. 3, indicating the relative amounts of gasoline, jet fuel, and diesel boiling-range hydrocarbons. A representative FT gas product composition is provided in Table 3. This can be combusted in the reformer to provide heat energy or otherwise recycled to extinction.

TABLE 3

| | Mole % |
|---|---|
| H₂ | 59.9 |
| CO | 24.2 |
| CO₂ | 4.34 |
| methane | 8.91 |
| ethane | .43 |
| ethylene | .02 |
| propane | .63 |
| propylene | .06 |
| butanes | .53 |
| butene | .18 |
| pentanes | .26 |
| pentenes | .17 |
| C₆⁺ | .35 |
| total | 100 |

### EXAMPLE 3

### Reverse Water-Gas Shift (RWGS)

The catalyst used in Example 1 for performing the reforming reaction was additionally effective for carrying out the RWGS reaction. At low temperature, equilibrium is more favorable to methanation and low CO production. In order to achieve high CO₂ conversion to CO, the RWGS reaction must be performed at a sufficiently high temperature. The Pt/Rh on cerium oxide catalyst attained 75% CO₂ conversion to CO at 913° C. (1675° F.) and a gauge pressure of 103 kPa (15 psig), with a 2.9 H₂:CO molar ratio in the product synthesis gas. The experimental results are summarized in Table 4.

TABLE 4

| | CO₂ + H₂ | | CO₂ + H₂ | |
|---|---|---|---|---|
| | Feed 1 | Product 1 | Feed 2 | Product 2 |
| methane, mole % | | .2 | | .2 |
| ethane, mole % | | | | |
| propane, mole % | | | | |
| CO₂•Mole % | 24 | 7.2 | 31 | 7.8 |
| water, mole % | | 20.4 | | 10.0 |
| H₂, mole % | 76 | 53.8 | 69 | 61.2 |
| CO, mole % | | 18.4 | | 20.8 |
| % CO₂ conversion | | 75 | | 73 |
| H₂:CO molar ratio | | 2.92 | | 2.92 |
| gas flow rate cc/min | 615 | 440 | 920 | 750 |
| wt-% recovery | | 89 | | 85 |

Whereas the 2.9 H₂:CO molar ratio in the product gas was high for a typical FT feed, this ratio is reduced to 2.1 when combined with recycled FT gas product, according to modeling studies.

### EXAMPLE 4

### Electric Reformer Studies

Reforming tests were conducted using an electrically-heated reformer, with the catalyst as described in Example 1 and with an “IH² type feed” having a composition of approximately 24 mol-% H2, 17.5 mol-% methane, 17.5 mol-% propane, and 41 mol-% CO₂. The electric reactor heaters were placed in a sheath that protected them from exposure to the process gas and also facilitated changeout in case of a heater burn out. Typical experimental parameters, for the preparation of synthesis gas from the gaseous feed mixture, and results are summarized in Table 5.

TABLE 5

| | |
|---|---|
| temperature, ° C. (top- internal) | 810 |
| temperature, ° C. ( top sheath) | 900 |
| pressure, psig | 50 |

FEED TO REFORMER

| | |
|---|---|
| water, g/hr | 996 |
| gas, g/hr | 1232 |
| gas, l/min | 15.9 |
| total, g/hr | 2228 |
| WHSV | 1.3 |
| moles steam/moles carbon | .96 |
| (including CO + CO₂) | |
| moles steam/mole carbon | 1.9 |
| (excluding CO + CO₂) | |

PRODUCT FROM REFORMER

| | |
|---|---|
| water collected, g/hr | 440 |
| gas, g/hr | 1671 |
| Total, g/hr | 2111 |

PRODUCT COMPOSITION, mole %

| | |
|---|---|
| H₂, mole % | 60.9 |
| CO, mole % | 24.5 |
| CO₂, mole % | 12.4 |
| Methane, mole % | 2.2 |
| gas, l/min | 45 |
| H₂:CO molar ratio | 2.49 |
| % hydrocarbon conversion | 90 |

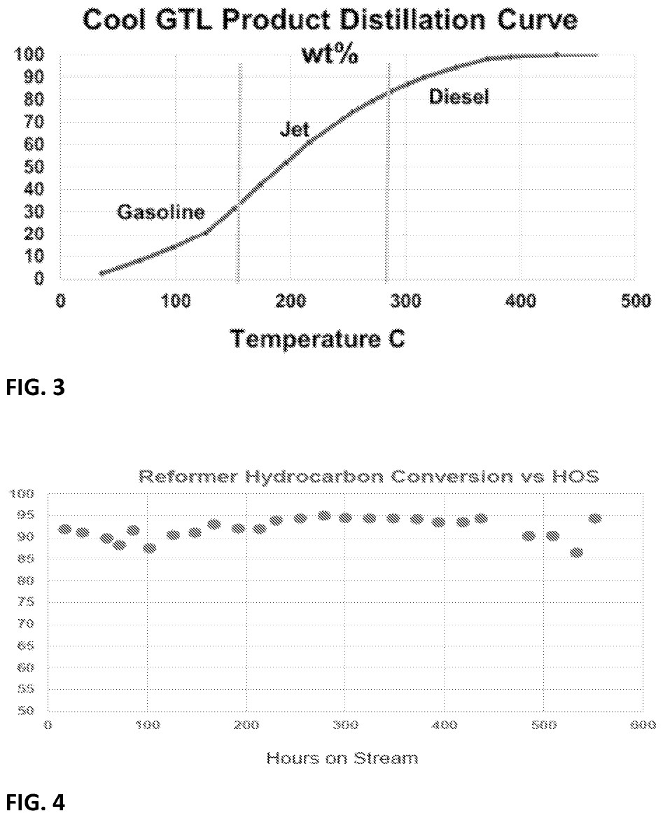

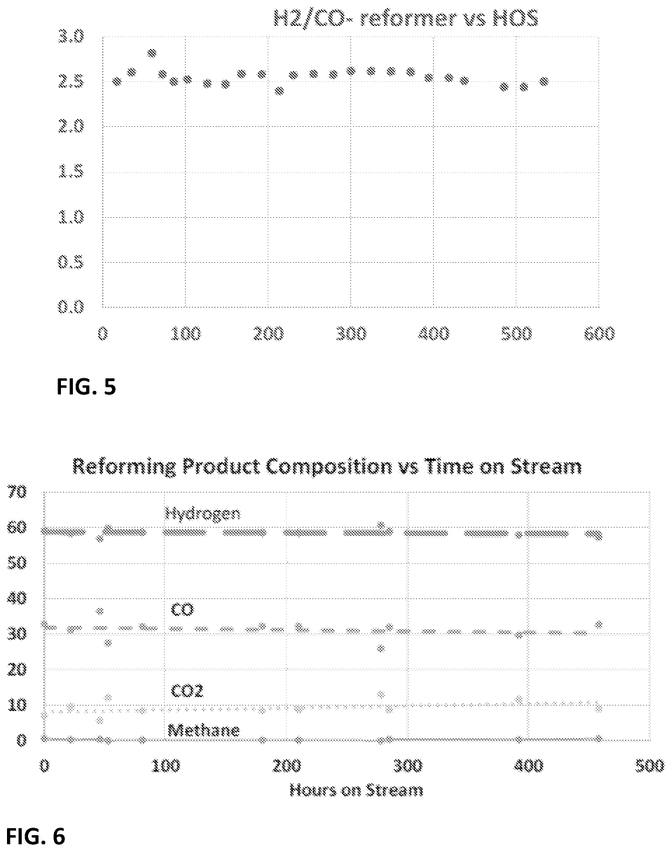

In total, the electric reformer was operated for more than 500 hours in a reliable manner, and without any problems associated with increasing WHSV from 0.7 to 1.5 hr⁻¹. The reformer hydrocarbon conversion was generally 90-95% over the operating period, as shown in FIG. 4, and the H₂:CO molar ratio of the synthesis gas produced, as shown in FIG. 5, was also consistent over this period. Although this molar ratio was measured as low as 2.4, the average value was higher, and in practice adjustments can be made to this ratio as needed. The reforming product composition over time was also stable, as shown in FIG. 6. The material balance over the operating period centered around 100%, providing an indication of reliability of the measured parameters. Overall, the results demonstrated that the electric reformer performed well over an extended operation, for production of synthesis gas from mixture comprising methane and CO₂, which would be expected to provide favorable outcomes for a wide variety of feeds, such as biogas.

Overall, aspects of the invention relate to processes that utilize reforming and/or RWGS reactions to convert low value gaseous feed mixtures to liquid hydrocarbon products, for example those comprising C₄⁺ hydrocarbons having carbon that is derived from renewable sources, such as CH₄ and CO₂ (that are the main components of biogas), and/or otherwise electrolysis H₂, fossil hydrogen with CCS, bio-gasification hydrogen, or methane pyrolysis hydrogen, and CO₂ derived from direct air capture, the gasification of biomass, or the combustion of biomass. Additional processing steps may optionally include FT synthesis alone or in combination with cracking to achieve a desired hydrocarbon molecular weight distribution.

Those having skill in the art, with the knowledge gained from the present disclosure, will recognize that various changes can be made to these processes in attaining these and other advantages, without departing from the scope of the present disclosure. As such, it should be understood that the features of the disclosure are susceptible to modifications and/or substitutions without departing from the scope of this disclosure. The specific embodiments illustrated and described herein are for illustrative purposes only, and not limiting of the invention as set forth in the appended claims.

1. A process for producing a liquid hydrocarbon product comprising C₄⁺ hydrocarbons, the process comprising:
(a) in a reforming stage or an RWGS stage, contacting a gaseous feed mixture comprising predominantly (i) H₂ and CO₂ or (ii) a hydrogen source and CO₂ with a reforming/RWGS catalyst to produce a synthesis gas intermediate comprising an H₂/CO mixture; and
(b) in a Fischer-Tropsch (FT) synthesis stage, converting the synthesis gas intermediate to said liquid hydrocarbon product, at least partially via FT synthesis.

2. The process of claim 1, wherein the gaseous feed mixture comprises (i) H₂ and CO₂ in a combined amount of at least about 75 mol-% or (ii) the hydrogen source and CO₂ in a combined amount of at least about 75 mol-%.

3. The process of claim 1, wherein the gaseous feed mixture comprises one or more of CO, H₂O, and O₂, independently in an amount, or in a combined amount, of less than about 10 mol-%.

4. The process of claim 1, wherein the gaseous feed mixture comprises biogas.

5. The process of claim 1, wherein the C₄⁺ hydrocarbons comprise naphtha boiling-range hydrocarbons, jet fuel boiling-range hydrocarbons, and/or diesel boiling-range hydrocarbons in a combined amount of at least about 80 wt-%.

6-24. (canceled)

25. A liquid hydrocarbon product comprising naphtha boiling-range hydrocarbons, jet fuel boiling-range hydrocarbons, and/or diesel boiling-range hydrocarbons having a renewable carbon content of at least about 70%.

26. The liquid hydrocarbon product of claim 25, wherein at least about 20% of a total carbon content of the liquid hydrocarbon product is derived from CO₂.

27. The liquid hydrocarbon product of claim 26, wherein said CO₂ is contained in biogas.

28. A recovered C₄⁺ hydrocarbon fraction comprising substantially all naphtha boiling-range hydrocarbons, jet fuel boiling-range hydrocarbons, and/or diesel boiling-range hydrocarbons, wherein (i) at least about 20% of a total carbon content of said recovered C₄⁺ hydrocarbon fraction is derived from atmospheric CO₂ and/or (ii) at least about 20% of a total hydrogen content of said recovered C₄⁺ hydrocarbon fraction is derived from electrolysis hydrogen.

29. A process for producing a liquid hydrocarbon product comprising naphtha boiling-range hydrocarbons, jet fuel boiling-range hydrocarbons, and/or diesel boiling-range hydrocarbons, the process comprising:
(a) in a reforming stage or an RWGS stage, contacting a gaseous feed mixture comprising predominantly (i) H₂ and CO₂ or (ii) a hydrogen source and CO₂ with a reforming/RWGS catalyst to produce a synthesis gas intermediate comprising an H₂/CO mixture; and
(b) in a Fischer-Tropsch (FT) synthesis stage, converting the synthesis gas intermediate to said liquid hydrocarbon product, via Fischer-Tropsch (FT) synthesis in combination with wax cracking.

30. The process of claim 29, wherein step (b) comprises contacting the H₂/CO mixture with a mixture of an FT catalyst and a cracking catalyst, to provide an FT synthesis effluent.

31. The process of claim 30, wherein the cracking catalyst comprises one or more cracking active metals selected from Groups 12-14 of the Periodic Table.

32. The process of claim wherein step (b) further comprises contacting the FT synthesis effluent with a polishing catalyst in the substantial absence of the FT catalyst, to provide a polishing effluent.

33. The process of claim 32, wherein step (b) comprises contacting the H₂/CO mixture with a bi-functional catalyst having an FT-functional constituent and a cracking-functional constituent, to provide an FT synthesis effluent.

34. The process of claim 33, wherein the cracking-functional constituent comprises one or more cracking active metals selected from Groups 12-14 of the Periodic Table.

35. The process of claim wherein step (b) further comprises contacting the FT synthesis effluent with a polishing catalyst in which the FT-functional constituent is substantially absent, to provide a polishing effluent.

36. The process of claim wherein, in step (b), the polishing catalyst is contacted with the FT synthesis effluent in addition to a fresh makeup H₂-containing feed, comprising at least about 50 mol-% H₂ and provided directly to the polishing catalyst.

37. The process of claim 1, active metals are deposited on a solid acidic support. wherein the one or more cracking

38. The process of claim 37, wherein the solid acidic support comprises a zeolitic molecular sieve having a silica to alumina molar framework ratio of less than about 50.

39. The process of claim 1, wherein the liquid hydrocarbon product is contained in an FT synthesis effluent, wherein the process further comprises:
separating the liquid hydrocarbon product comprising the C₄⁺ hydrocarbons from the FT synthesis effluent, and
separating a fraction enriched in (i) H₂ and CO₂ or (ii) the hydrogen source and CO₂, from the FT synthesis effluent.

40-60. (canceled)
